(19) United States
(12) Patent Application Publication
(10) Pub. No.: US 2024/0358384 A1
(43) Pub. Date: Oct. 31, 2024

(54) Medical Device With Acoustic Sensor(s) and Method for Localizing Medical Device and Acoustic Source

(71) Applicant: Applaud Medical, Inc., San Francisco, CA (US)

(72) Inventors: Yuri A. Pishchalnikov, Rohnert Park, CA (US); Rohit Girotra, San Francisco, CA (US); Michal Jenco, San Carlos, CA (US); Matthew Hopcroft, San Francisco, CA (US); Onofre Jauregi Ruano, Fillmore, CA (US); Mina Wai Bing Chow, Campbell, CA (US)

(21) Appl. No.: 18/645,970

(22) Filed: Apr. 25, 2024

Related U.S. Application Data
(60) Provisional application No. 63/498,297, filed on Apr. 26, 2023.

(51) Int. Cl. A61B 17/22; A61B 8/08; A61B 8/12; B06B 1/06

(52) U.S. Cl. CPC A61B 17/22012; A61B 8/085; A61B 8/12; A61B 8/48; B06B 1/0688; A61B 2017/22014; B06B 2201/76

(57) ABSTRACT

A catheter includes a shaft; a tip disposed at a distal end of the shaft; at least one acoustic sensor disposed on or in the shaft, each acoustic sensor disposed at a respective distance from the distal end of the shaft; and at least one electrical conductor disposed on or in the shaft, each electrical conductor electrically connecting a respective acoustic sensor to one or more electrical connection points in a housing attached to a proximal end of the shaft. The catheter and an acoustic source are localized with respect to each other using acoustic signals transmitted between the acoustic source and the catheter.

### CROSS-REFERENCE TO RELATED APPLICATIONS

This application claims priority to U.S. Provisional Application No. 63/498,297, filed on Apr. 26, 2023, titled “System and Method for Positioning a Treatment Apparatus With Respect to a Catheter,” which is hereby incorporated by reference.

### TECHNICAL FIELD

This application relates generally to a medical device having one or more acoustic sensors and localizing an acoustic source and such a medical device with respect to each other.

### BACKGROUND

Many treatment procedures require positioning and monitoring the location of a catheter in a vessel. Although X-ray imaging can allow visualization of the catheter, alternative approaches without the use of ionizing radiation are preferable. Ultrasound scanners can provide nonionizing visualization, but the image resolution can be challenging, making it difficult to reliably identify and monitor the position of the catheter. For example, image resolution is better at higher frequencies which has poor penetration depth, thus making ultrasound imaging difficult at larger depths. It would be desirable to improve the ability to localize and monitor the position of the catheter without the use of ionizing radiation.

For many treatment procedures it would be desirable to use the position of the catheter as a marker, e.g., for the delivery of electromagnetic or acoustic energy into a specific region in a human body. The delivery of acoustic energy to the target is typically associated with various challenges. One challenge is alignment of the ultrasound beam on the target, which is also affected by diffraction and deflection of acoustic waves passing through different layers of tissue along the acoustic path to the target. Another challenge is acoustic coupling between the acoustic source and the skin of a patient, as well as ensuing an acoustic window in a patient body for efficient passage of acoustic waves to the target.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to a catheter comprising a shaft; a tip disposed at a distal end of the shaft; at least one acoustic sensor disposed on or in the shaft, each acoustic sensor disposed at a respective distance from the distal end of the shaft; and at least one electrical conductor disposed on or in the shaft, each electrical conductor electrically connecting a respective acoustic sensor to one or more electrical connection points in a housing attached to a proximal end of the shaft.

In one or more embodiments, each acoustic sensor comprises a piezoelectric polymer film disposed about at least a portion of a circumference of the shaft. In one or more embodiments, the piezoelectric polymer film comprises polyvinylidene fluoride.

In one or more embodiments, the shaft includes an inner tube and an outer tube, the respective piezoelectric polymer film is disposed about at least a portion of a circumference of the inner tube, and the at least one electrical conductor is disposed between the inner and outer tubes. In one or more embodiments, the inner tube is defined by a wall having an inner-wall thickness, and one or more regions of the wall have an increased thickness compared to the inner-wall thickness. In one or more embodiments, the outer tube is defined by a wall having an outer-wall thickness, and one or more regions of the wall have an increased thickness compared to the outer-wall thickness. In one or more embodiments, a spacer is disposed between the inner and outer tubes.

In one or more embodiments, the one or more electrical connection points is/are electrically coupled to a cable that extends through the housing. In one or more embodiments, the one or more electrical connection points is/are formed on a printed circuit board disposed in the housing. In one or more embodiments, the catheter further comprises wireless communication circuitry electrically coupled to the one or more electrical connection points.

In one or more embodiments, the housing includes a port having a hole that is aligned with a central channel of the shaft. In one or more embodiments, the at least one acoustic sensor includes first and second acoustic sensors, the first and second sensors separated by a predetermined distance.

Another aspect of the invention is directed to a method for localizing an acoustic source and a medical device with respect to each other, the method comprising a. introducing the medical device into a mammal; b. acoustically coupling the acoustic source to the mammal at a position that corresponds to a target location of the medical device, the acoustic source comprising a housing and a source transducer disposed in the housing; c. producing acoustic signals with the source transducer; d. receiving the acoustic signals with an acoustic sensor on or in the medical device, the acoustic sensor in electrical or wireless communication with a detector; e. determining, with the detector, a time-of-flight (ToF) of the acoustic signals transmitted between the source transducer and the acoustic sensor; f. determining, with the detector and using the ToF, a distance between the source transducer and the acoustic sensor; and g. localizing, with the detector, the acoustic source and the medical device with respect to each other in real time based, at least in part, on the distance between the source transducer and the acoustic sensor.

In one or more embodiments, the acoustic sensor is a first acoustic sensor, the medical device includes at least a second acoustic sensor, and the method further comprises receiving the acoustic signals with the first and second acoustic sensors; determining, with the detector, a first ToF of the acoustic signals transmitted between the source transducer and the first acoustic sensor; determining, with the detector, a second ToF of the acoustic signals transmitted between the source transducer and the second acoustic sensor; determining, with the detector and using the first ToF, a first distance between the source transducer and the first acoustic sensor; determining, with the detector and using the second ToF, a second distance between the source transducer and the second acoustic sensor; and localizing the acoustic source and the medical device with respect to each other based, at least in part, on the first and second distances.

In one or more embodiments, the medical device includes a plurality of acoustic sensors, the acoustic source includes a plurality of source transducers, and the method further comprises sequentially producing the acoustic signals with at least a first source transducer and a second source transducer of the plurality of source transducers; receiving the acoustic signals with each acoustic sensor; determining a respective ToF of the acoustic signals transmitted between (a) each of the at least the first source transducer and the second source transducer and (b) each acoustic sensor; determining, using each ToF, respective distances between (a) each of the at least the first source transducer and the second source transducer and (b) each acoustic sensor; and localizing the acoustic source and the medical device with respect to each other based, at least in part, on the respective distances.

In one or more embodiments, the position is a first position, and the method further comprises after performing at least steps b-f while the acoustic source is located at the first position, moving the acoustic source to a second position and repeating steps b-f while the acoustic source is located at the second position so as to improve a resolution of a localization of the acoustic source and the medical device with respect to each other compared to when the localization is performed while the acoustic source is only located at the first position.

In one or more embodiments, the medical device comprises a catheter, the catheter is introduced into an organ, and the method further comprises introducing, with the catheter, an acoustic enhancer proximal to a calcification; applying acoustic energy with the acoustic source; and producing cavitation with the acoustic energy and the acoustic enhancer to disintegrate at least a portion of calcification.

In one or more embodiments, the method further comprises adjusting a position of the acoustic source, with a robotic positioner in communication with the detector, according to a localization of the acoustic source and the medical device with respect to each other. In one or more embodiments, the method further comprises displaying relative positions of the acoustic source and the medical device on a display screen on or in electrical communication with the detector.

In one or more embodiments, the medical device comprises a catheter or a guidewire.

Another aspect of the invention is directed to a method for localizing an acoustic source and a catheter with respect to each other, the method comprising: a. introducing the catheter into a mammal, the catheter comprising a shaft and a tip disposed at a distal end of the shaft; and at least one acoustic sensor disposed on or in the shaft, each acoustic sensor dispose at a respective distance from the distal end of the shaft; b. acoustically coupling the acoustic source to the mammal at a position that corresponds to a target location of the catheter, the acoustic source comprising a housing and a plurality of source transducers disposed in the housing; c. producing a broad beam of acoustic energy with the acoustic source; d. determining, with a detector in electrical or wireless communication with the at least one acoustic sensor, a measured distance between the source transducers and the at least one acoustic sensor, the measured distance based, at least in part, on a ToF of the acoustic signals transmitted between the source transducers and each acoustic sensor; c. setting a focal distance for the source transducers corresponding to the measured distance; f. producing a focused beam of acoustic energy with the acoustic source while moving the acoustic source parallel to a first axis that is orthogonal to an acoustic axis of the acoustic transducers, the focused beam focused at the focal distance; g. monitoring, with the detector, output signals of the at least one acoustic sensor to determine a first maximum amplitude signal while the focused beam is produced, the first maximum amplitude representing a first localization with respect to the first axis; h. after step g, sweeping the focused beam with respect to a second axis that is orthogonal to an acoustic axis of the acoustic transducers, the ultrasound source located at a position corresponding to the first maximum amplitude signal; i. monitoring, with the detector, the output signals of the at least one acoustic sensor to determine a second maximum amplitude signal while the focused beam is swept, the second maximum amplitude representing a second localization with respect to the second axis; j. after step h, rotating the focused beam with respect to the acoustic axis; and k. monitoring, with the detector, the output signals of the at least one acoustic sensor to determine a third maximum amplitude signal while the focused beam is rotated, the third maximum amplitude representing a third localization with respect to the acoustic axis.

In one or more embodiments, the method further comprises locking the acoustic source at the position corresponding to the first maximum amplitude signal.

Another aspect of the invention is directed to a guidewire comprising a core; a coil coaxially disposed over the core; a protective coating disposed on the coil; at least one acoustic sensor disposed at a respective distance from a distal end of the shaft; and at least one electrical conductor disposed in the protective coating, each electrical conductor electrically connecting a respective acoustic sensor to one or more electrical connection points in a housing attached to a proximal end of the guidewire.

In one or more embodiments, each acoustic sensor comprises a piezoelectric polymer film disposed about at least a portion of a circumference of the core. In one or more embodiments, each acoustic sensor comprises a piezoelectric polymer film disposed about at least a portion of a circumference of the protective film. In one or more embodiments, each acoustic sensor comprises a piezoelectric polymer film disposed in the protective film.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 is a block diagram of a system for acoustically localizing an acoustic source with respect to a catheter according to an embodiment.

FIG. 2 is an isometric view of the catheter illustrated in FIG. 1 according to an embodiment.

FIG. 3 is an enlarged view of a distal end of the catheter illustrated in FIG. 2.

FIGS. 4A-C are a cross sections of the shaft of the catheter by a first plane according to different embodiments.

FIG. 5 is a cross section of the shaft of the catheter by a second plane according to an embodiment.

FIGS. 6A-D are cross sections of the shaft of the catheter by a third plane according to different embodiments.

FIG. 7 is a top view of the housing of the catheter illustrated in FIG. 2 with the cover removed.

FIG. 8 is a flow chart of a method for localizing an acoustic source and a catheter with respect to each other.

FIG. 9 illustrates an example localization method according to an embodiment.

FIG. 10 illustrates an example localization method according to another embodiment.

FIG. 11 is a flow chart of a method for localizing an acoustic source and a catheter with respect to each other according to another embodiment.

FIG. 12 shows an example coordinate system.

FIGS. 13A and 13B show example traces of two acoustic sensors on a catheter and their respective differential signals.

FIG. 14 shows example traces from two acoustic sensors on a catheter oriented along the acoustic axis of the acoustic source.

FIG. 15 is a block diagram of a system for acoustically localizing an acoustic source with respect to a catheter according to another embodiment.

FIG. 16 is a partially transparent side view of a guidewire according to an embodiment.

FIG. 17A and 17B are cross sections of the guidewire illustrated in FIG. 16 according to different embodiment.

FIG. 18 is a partially transparent side view of a guidewire according to another embodiment.

FIG. 19A-C are cross sections of the guidewire illustrated in FIG. 18 according to different embodiments.

FIG. 20 is a block diagram of a system for acoustically localizing an acoustic source with respect to a guidewire according to an embodiment.

FIG. 21 shows an example embodiment in which a medical device with multiple acoustic sensors is inserted through the ureter in the collecting system of the human kidney.

### DETAILED DESCRIPTION

A catheter includes a shaft having one or more acoustic sensors disposed on or in the shaft and at respective distance(s) from a distal end of the shaft. The acoustic sensor(s) can include a respective piezoelectric polymer film that is disposed about some or all of the circumference of an inner tube or of an outer tube of the shaft. The outer tube is disposed over the inner tube to cover the acoustic sensor(s). Leads and wires for the acoustic sensors can be disposed between the inner and outer tubes.

A guidewire includes a coil, a core, and having one or more acoustic sensors disposed on or in the guidewire at respective distance(s) from a distal end of the guidewire. The acoustic sensor(s) can include a respective piezoelectric polymer film that is disposed about some or all of the circumference of a protective coating of the coil or about some or all of the circumference of the core.

The acoustic sensor(s) can be used to localize the catheter or guidewire and an acoustic source with respect to each other, without imaging, after the catheter or guidewire is inserted or introduced into a mammal.

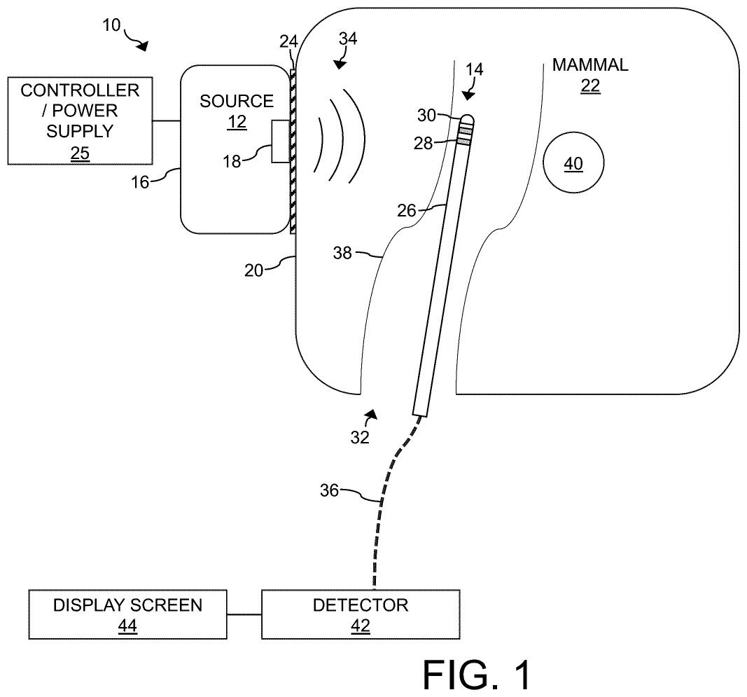

FIG. 1 is a block diagram of a system 10 for acoustically localizing an acoustic source 12 with respect to a catheter 14 according to an embodiment. The acoustic source 12 includes a housing 16 and one or more acoustic transducers 18 disposed in the housing 16. The acoustic source 12 can comprise an acoustic treatment head that can be configured to produce acoustic energy to perform therapy or a medical procedure. The acoustic source 12 can be placed on the skin 20 of a mammal 22 such as a human. An acoustically transmitting media 24, such as water, a water cushion, an acoustic coupling oil, and/or an acoustic coupling gel, can be disposed between (e.g., in direct physical contact with) the acoustic source 12 and the skin 20 to improve acoustic transmission. The acoustic source 12 can be powered by a power supply, an amplifier, and/or a controller 25 that is electrically coupled to the acoustic source 12.

The catheter 14 includes a shaft 26 and one or more acoustic sensors 28 disposed on or in the shaft 26. The acoustics sensor(s) 28 can be located at predetermined position(s) from a distal end of the shaft 26 and/or from a tip 30 at the distal end of the catheter 14. After the catheter 14 is introduced into the mammal 22 such as a through a natural or surgical opening 32, the acoustic source 12 and the catheter 14 can be localized and/or aligned with respect to each other using acoustic signals 34 produced by the acoustic source 12 and received by the acoustic sensor(s) 28 on the catheter 14. For example, the time-of-flight (ToF) and/or amplitude (e.g., maximum) of the acoustic signals 34 can be used to localize and/or align the acoustic source 12 and the catheter 14. The catheter 14 can be placed in an anatomical structure in the mammal 22, such as in an anatomical channel 38 (e.g., the urethra, the rectum, or a blood vessel), an internal organ, or another anatomical structure. The catheter 14 can be placed near a target volume 40 that may be the target of a therapeutic and/or a medical procedure. The catheter 14 can be used to introduce a guidewire, a tool, an acoustic enhancer (e.g., engineered microbubbles), fluids, and/or a therapeutic substance to or near the target volume 40. For example, an acoustic enhancer can be used to facilitate breakage of calcifications, such as urinary stone(s) or kidney stone(s), at low pressure amplitudes by promoting localized cavitation.

The acoustic sensor(s) 28 can be electrically coupled (e.g., via a cable or wire(s) 36 (in general, cable)) to a detector 42 to detect and/or analyze the acoustic signals (e.g., acoustic signals 34) received by the acoustic sensor(s) 28, which have been converted by the acoustic sensor(s) 28 to electrical signals. Additionally or alternatively, the electrical signal data, representing the acoustic signals received by the acoustic sensor(s) 28, can be transmitted wirelessly to the detector 42. The wireless transmission can be performed using a local wireless protocol such as Bluetooth, a local wireless network such as WiFi, a wide area wireless network such as a cellular network, or another wireless transmission network or protocol.

The detector 42 can comprise a computer, a treatment console (e.g., a portion of a treatment console), a data acquisition board, an oscilloscope, or any other device to precondition and/or detect the acoustic signals received by the acoustic sensor(s) 28. In some embodiments, the detector 42 and the controller/power supply 25 can be combined, for example in a treatment console.

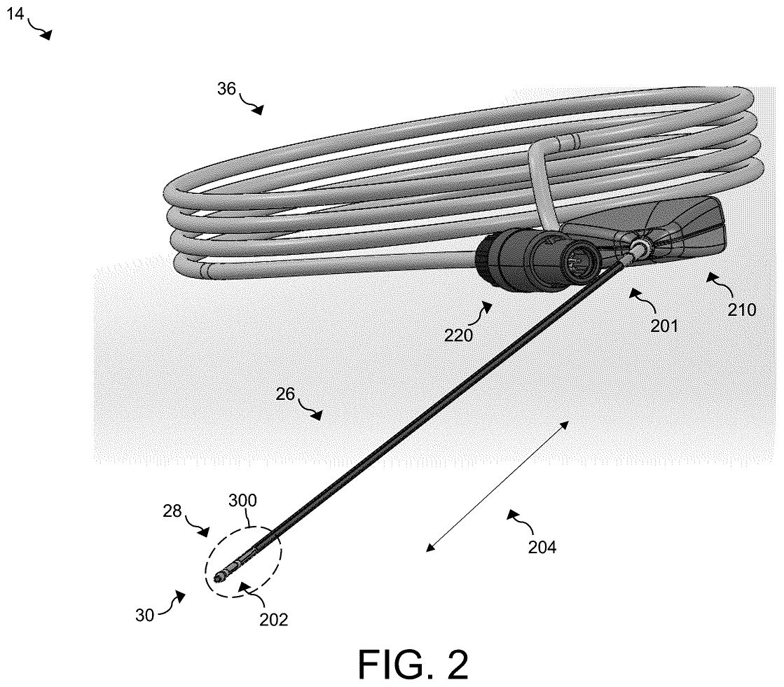

FIG. 2 is an isometric view of the catheter 14 according to an embodiment. The shaft 26 has a proximal end 201 and a distal end 202. The shaft 26 can extend from the proximal end 201 to the distal end 202 parallel to an axis 204. The shaft 26 can be flexible and/or bendable such that the shaft 26 includes one or more curves and/or bends.

A housing 210 is disposed and/or attached to the proximal end 201 of the shaft 26. The housing 210 can also be referred to as a connecting hub or a handle. The housing 210 can enclose electrical connections between the acoustic sensor(s) 28 and the cable 36. A proximal end of the cable 36 can include an electric plug 220 that can be electrically connected to a detector 42. The housing 210 can also include one or more ports to connect with respective channel(s) in the shaft 26.

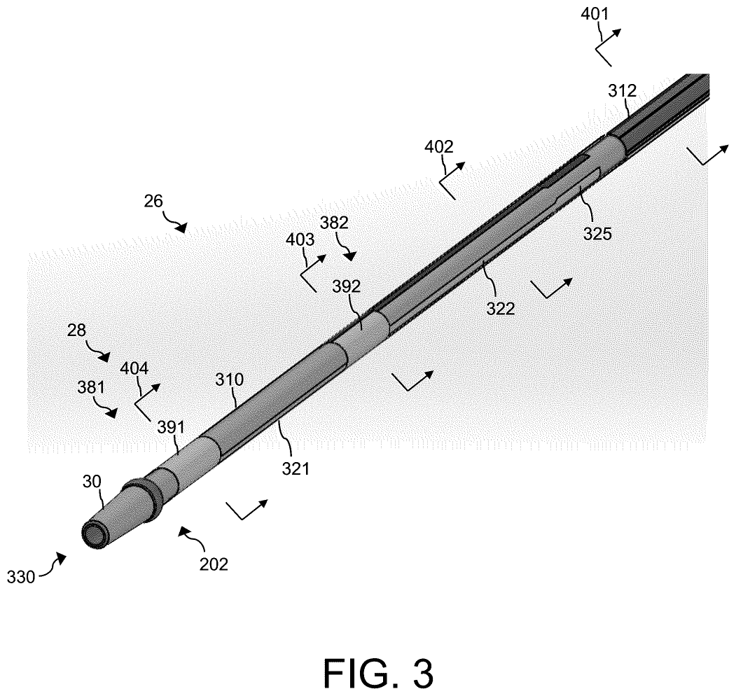

FIG. 3 is an enlarged view of region 300 in FIG. 2 which corresponds to the distal end of the catheter 14. In this embodiment, the acoustic sensor(s) 28 include a first acoustic sensor 381 and a second acoustic sensor 382. In other embodiments, the acoustic sensor(s) only include a first acoustic sensor 381 or a second acoustic sensor 382. In other embodiments, the acoustic sensor(s) 28 include more than two acoustic sensors.

The first acoustic sensor 381 includes a first piezoelectric polymer film 391 wrapped around and/or disposed on an inner tube 310 of the shaft 26. The second acoustic sensor 382 includes a second piezoelectric polymer film 392 wrapped around and/or disposed on the inner tube 310 of the shaft 26. The shaft 26 includes an outer tube 312 that is disposed over the inner tube 310. For illustration purposes only, the outer tube 312 is illustrated as not extending to the distal end 202 of the shaft 26 to not obscure the first and second sensors 381, 382. However, the outer tube 312 may extend to the distal end 202 of the shaft 26 (e.g., to a proximal end of the tip 30). The outer tube 312 can be configured to cover the first and second sensors 381, 382, including the first and second piezoelectric polymer films 391, 392, such that the first and second sensors 381, 382 (and the first and second piezoelectric polymer films 391, 392) are between the inner tube 310 and the outer tube 312. The inner tube 310 and the outer tube 312 are coaxial.

Electrical leads 321, 322 are disposed on the inner tube 310. A distal end of each electrical lead 321, 322 is electrically connected to a respective one or both of the first and second sensors 381, 382 (e.g., to one or both of the first piezoelectric polymer film 391 and/or the second piezoelectric polymer film 392, respectively). A proximal end of each electrical lead 321, 322 is electrically connected to one or more electrical contact pads 325. In some embodiments, the proximal end of each electrical lead 321, 322 is electrically connected to a respective electrical contact pad 325. In some embodiments, there can be more than two electrical leads 321, 322, such as three or more electrical leads electrically connected to one or more electrical contact pads 325. In an embodiment, there are three electrical leads (e.g., including electrical leads 321, 322) and three electrical pads 325, where each lead is electrically connected to a respective electrical pad 325. Electrical wires can be electrically connected to the electrical pads 325 and an electrical connection point in the housing 210. The electrical wires can be disposed between the inner tube 310 and the outer tube 312.

The first and second piezoelectric polymer films 391, 392 comprise or consist of a piezoelectric polymer such as polyvinylidene fluoride (PVDF), Pb (Zr,Ti) O3 (PZT), AlN Poly (vinylidene fluoride-trifluoroethylene) (PVDF-TrFE) copolymers, BaTiO3/PVDF-TrFE, ZnO films, Zr₂P₂BrCl, M₂CO₂, and/or another piezoelectric polymer. A piezoelectric polymer, such as PVDF, is a polarized fluoropolymer that, in its polar form (e.g., β-phase), demonstrates strong and stable piezoelectric and pyroelectric activities. Ferroelectricity is the property of having a spontaneous electric polarization that can be reversed by the application of an external electric field. This property is typically observed in materials with a non-centrosymmetric crystal structure that allows dipoles to be formed and switched within the crystal.

Piezoelectric polymer films, such as those comprising or consisting of PVDF, can provide ultrasonic performance at a low cost with consistent unit-to-unit repeatability. PVDF films can provide broad frequency bandwidths with low Q-factors and low electrical impedance (e.g., 30-100 Ohms). In addition, PVDF films are lightweight and flexible to conform with the cylindrical surface of the catheter 14 (e.g., of the inner tube 310). PVDF films can also provide excellent acoustic matching with liquids and/or biological tissues.

PVDF films are mechanically robust and are also used in structural and coating applications without specific need for piezoelectric properties. In an embodiment, the shaft 26 (or a portion of the shaft 26) can be formed out of PVDF (or another piezoelectric polymer material) and the acoustic sensor(s) 28 can be formed by depositing electrodes on certain locations of the PVDF structure.

The enlarged view also illustrates that the tip 30 can be tapered, for example, to facilitate easy passage of the catheter 14 along or through an anatomical feature such as a vessel (e.g., a blood vessel) or a ureter. The tip 30 includes an opening 330 that is coupled to one or more channels defined in the inner tube 310.

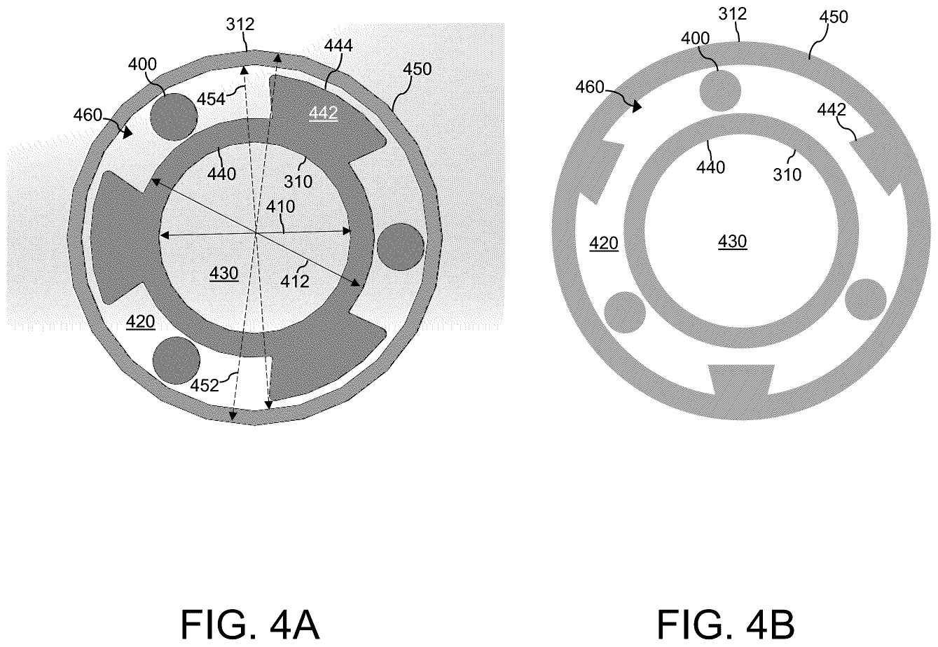

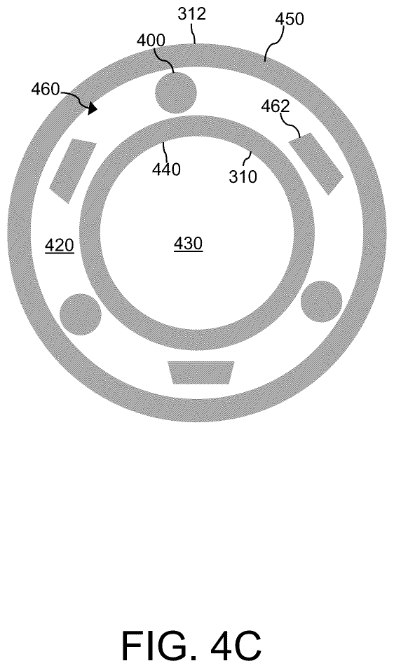

FIG. 4A is a cross section of the shaft 26 by plane 401 in FIG. 3. The cross section is the shaft 26 between the electrical pads 325 and the proximal end 201 of the shaft 26. The cross section illustrates that one or more (e.g., a plurality of) wires 400 or other electrical conductors are disposed between the inner tube 310 and the outer tube 312. In the illustrated embodiment, there are three wires 400. In other embodiments, there can be only one wire 400, two wires 400, four wires 400, or another number of wires. The wires 400 can be electrically connected to respective electrical pads 325. In some embodiments, a wire 400 can be electrically connected to more than one electrical pad 325 and/or multiple wires 400 can be electrically connected to the same electrical pad 325.

Wires 400 can be either electrically insulated or exposed wires. In addition, an insulator material 420 can be disposed between the inner tube 310 and the outer tube 312 and can surround each wire 400 to provide electrical insulation thereto. The insulator material 420 can also improve the mechanical strength of the shaft 26.

One or more channels 430 is/are defined by at least an inner diameter 410 of the inner tube 310. The channel(s) 430 can be used to introduce a guidewire, a tool, an acoustic enhancer (e.g., engineered microbubbles), fluids, and/or a therapeutic substance to or near a target volume. The channel(s) 430 extend to the opening 330 at the tip 30.

The inner tube 310 has a wall 440 having an inner-tube thickness that can be defined by the difference between an outer diameter 412 and the inner diameter 410 of the inner tube 310. The radial thickness of the wall 440 can have one or more localized regions 442 of increased thickness compared to the inner-tube thickness. For example, localized segments 444 of the wall 440 can have a larger localized outer diameter or outer radius compared to the outer diameter 412 (or outer radius). The localized regions 442 can be regions (e.g., extrusion regions) where additional material forming the wall 400 is extruded during manufacturing. The localized regions 442 increase the structural strength and/or rigidity of the inner tube 310 and can increase the structural strength and/or rigidity of the shaft 26 as a whole. Additionally or alternatively, the localized regions 442 can be disposed between neighboring wires 400 to physically separate and electrically isolate the wires 400.

The outer tube 312 has a wall 450 having an outer-tube thickness that can be defined by the difference between an outer diameter 452 and an inner diameter 454 of the outer tube 312. A gap 460 is defined between the outer diameter 412 of the inner tube 310 and the inner diameter 454 of the outer tube 312. The wire(s) 400, the insulator material 420, and the localized regions 442 are disposed in the gap 460.

In other embodiments, the localized regions 442 can be on the wall 450 of the outer tube 312 instead of on the wall 440 of the inner tube 310, for example as illustrated in FIG. 4B, which is a cross section of the shaft 26 by plane 401 in FIG. 3 according to another embodiment. The localized regions 442 increase the structural strength and/or rigidity of the outer tube 312. The cross section illustrated in FIG. 4B is the same as the cross section illustrated in FIG. 4A except for the location of the localized regions 442, and thus not all features of the cross section illustrated in FIG. 4B are labelled as they are in FIG. 4A. In other embodiments, the localized regions 442 can be on both the wall 450 of the outer tube 312 and the wall 440 of the inner tube 310.

In other embodiments, the localized regions 442 can be replaced by spacers 462 that are constructed as an independent element and are not necessary a constituent part of the wall 450 of the outer tube 312 and/or of the wall 440 of the inner tube 310, as illustrated in FIG. 4C. The number, geometry, and position of the localized regions 442 or spacers 462 can vary, as would be apparent for those skilled in the art. Alternatively, the localized regions 442 or spacers 462, or both can be removed.

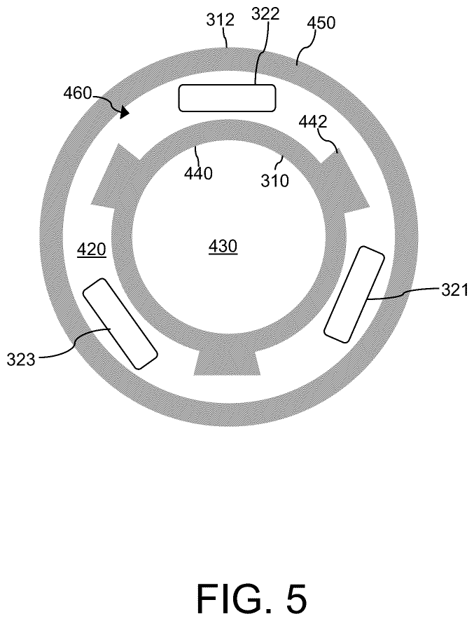

FIG. 5 is a cross section of the shaft 26 by plane 402 in FIG. 3. The cross section illustrated in FIG. 5 shows three electrical leads 321-323 disposed in the gap 460 between the wall 450 of the outer tube 312 and the wall 440 of the inner tube 310. There can be additional or fewer electrical leads 321-323 in other embodiments. The localized regions 442 are shown as being disposed on the wall 440 but can be on the wall 450 instead of or in addition to the wall 440, as discussed above. Alternatively, the localized regions 442 can be replaced with spacers 462 or can be removed.

The cross section illustrated in FIG. 5 is the same as the cross section illustrated in FIG. 4A except that the wires 400 are replaced with electrical leads 321-323, and thus not all features of the cross section illustrated in FIG. 5 are labelled as they are in FIG. 4A.

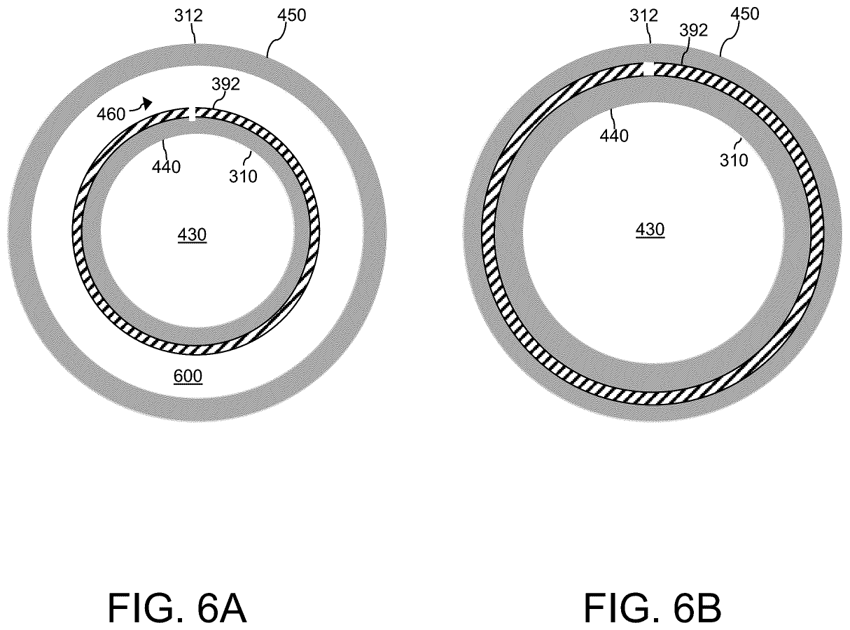

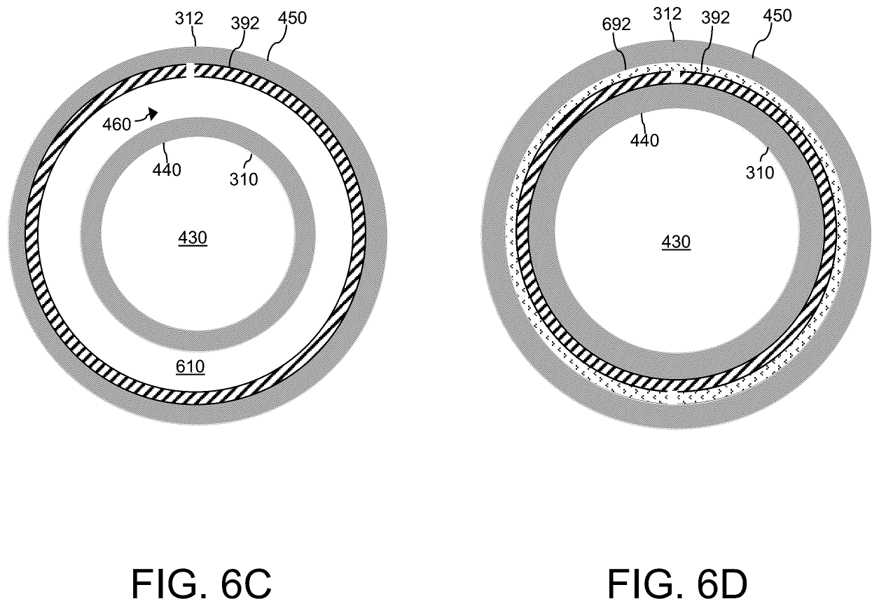

FIG. 6A is a cross section of the shaft 26 by plane 403 in FIG. 3. The cross section illustrated in FIG. 6A shows the second piezoelectric polymer film 392 disposed in the gap 460. The second piezoelectric polymer film 392 can be disposed about and/or cover the circumference of the wall 440 of the inner tube 310. In other embodiments, the second piezoelectric polymer film 392 can be disposed about and/or cover a portion (e.g., an arc) of the circumference of the wall 440, such in the range of about 30% to about 75%, including any values or ranges therebetween, of the circumference of the wall 440. The localized regions 442 and spacers 462 do not extend to this portion of the shaft 26 to allow the second piezoelectric polymer film 392 to be disposed about and/or cover the inner tube 310 without forming air gaps and/or to minimize air gaps. Good acoustic transmission requires an absence (or minimization) of air gaps between the outer tube 312 and the sensor (e.g., second piezoelectric polymer film 392). This can be achieved, for example, by filling the gap 460 with one or more materials 600 that have an acoustic impedance that matches (e.g., within about 20%) the impedance of bodily fluids and/or tissue, which have an acoustic impedance similar to that of water. In another embodiment, air gaps can be removed or reduced by the locally increased the diameter of the inner tube 310 so that the second piezoelectric polymer film 392 is in direct physical contact with the outer tube 312 as well as with the inner tube 310, as illustrated in FIG. 6B. In another embodiment, air gaps can be removed or reduced by disposing the second piezoelectric polymer film 392 about and/or cover the inner diameter of the outer tube 312, as illustrated in FIG. 6C. The gap 460 between the second piezoelectric polymer film 392 and the inner tube 310 can be filled with one or more materials 610 having a high acoustic impedance and/or that can provide an acoustically rigid interface to increase acoustic pressure at the acoustic sensor. In some embodiments, the material(s) 610 can be configured to absorb acoustic waves. Partial or complete absorption of acoustic waves (such as by an epoxy resin) can reduce or eliminate the reflection of acoustic waves, which may be desirable in some embodiments.

In some embodiments, two piezoelectric polymer films 392, 692 can be stacked and disposed about the wall 440 of the inner tube 310, for example as illustrated in FIG. 6D. One of the two piezoelectric polymer films can be reversed biased to reduce electromagnetic interference (EMI) and increase signal-to-noise ratio (SNR). The piezoelectric polymer films 392, 692 can be the same as or different than each other. To remove or reduce air gaps, the outer piezoelectric polymer film 692 can be in direct physical contact with the outer tube 312, as illustrated in FIG. 6B, or any gap between the outer piezoelectric polymer film 692 and the outer tube 312 can be filled with one or more materials 600 in the same manner as illustrated in FIG. 6A. Additionally or alternatively, any gap between the inner piezoelectric polymer film 392 and the inner tube 310 can be filled with one more materials 610 as illustrated in FIG. 6C. The inner and outer piezoelectric polymer films 392, 692 can be in direct physical contact with each other. The inner piezoelectric polymer film 392 can be in direct physical contact with the inner tube 310.

The localized regions 442 are shown as being disposed on the wall 440 but can be on the wall 450 instead of or in addition to the wall 440, as discussed above. Alternatively, the localized regions 442 can be replaced with spacers 462 or can be removed.

The cross sections illustrated in FIG. 6A-D are the same as the cross section illustrated in FIGS. 4A, 4B, or 4C except that the wires 400 are replaced with the piezoelectric polymer film 392 and the optional piezoelectric polymer film 692, and the insulator material 420 is replaced with acoustically transparent material(s) 600 or the gap 460 is removed by increasing the diameter of the inner tube 310, and thus not all features of the cross sections illustrated in FIG. 6A-D are labelled as they are in FIG. 4A-C.

A cross section through plane 404 in FIG. 3 can be the same as any of the cross sections illustrated in FIG. 6A-D except that in the cross section through plane 404 the second piezoelectric polymer film 392 is replaced with the first piezoelectric polymer film 391.

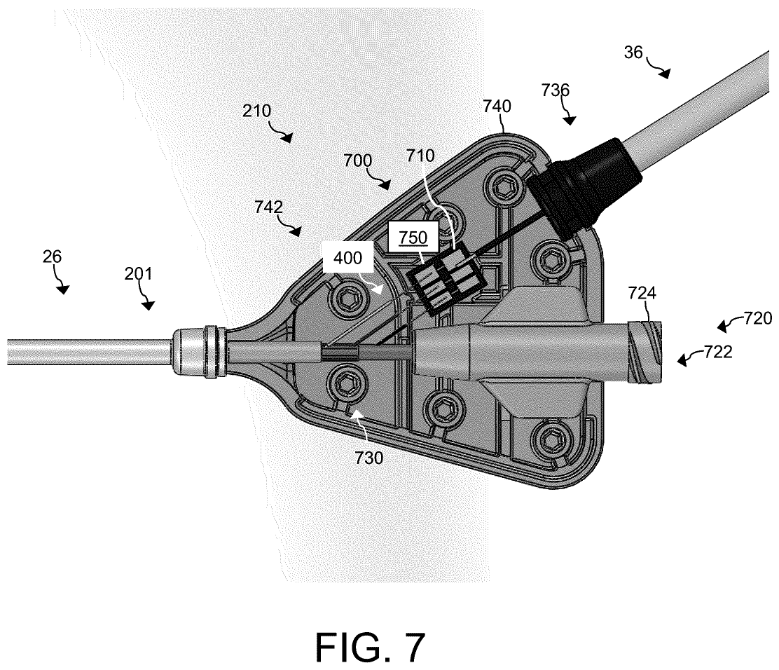

FIG. 7 is a top view of the housing 210 with a top cover removed to expose the inside of the housing 210. Electronics 700 are disposed in the housing 210. The electronics 700 can include devices with respective housings and/or electrical circuits mounted on a printed circuit board (PCB) 710. For example, the wires 400 that are electrically connected to the electrical contact pads 325 (FIG. 3) can be electrically connected to the electronics 700 (e.g., to the PCB 710). The electronics 700 can include electrical circuits to precondition the signal, improve the signal-to-noise ratio of the signals, and/or reject common-mode electromagnetic interference. In some embodiments, the electrical circuits can include one or more signal preamplifiers, common-mode chokes, and/or or common-mode rejection transformers. The wire connection (e.g., of the wires 400 to the electronics 700) in the housing 210 can be either direct to provide continuity for direct current (DC) or indirect with capacitive and/or inductive coupling to transmit only alternative current (AC). In addition, the electrical circuits can be used to electrically isolate the acoustic sensor(s) 28 from the detection circuitry (e.g., in an external detector such as detector 42 (FIG. 1)) by using an isolating device, such as, for example, isolation transformers or optical couplers.

An optional cable 36 can be electrically connected to the PCB 710 to receive the electrical signals after passing through the electronics 700. Thus, the housing 210 can provide a physical and electrical transition from the delicate wires 400 connecting the acoustic sensor(s) 28 to a more robust electric cable 36. The cable 36 can be connected to the housing using either another connector or permanently for example using a strain-relief device 736. Additionally or alternatively, the electrical signal data, representing the acoustic signals received by the acoustic sensor(s) 28, can be transmitted wirelessly using wireless communication circuitry 750 that can be mounted on and/or electrically coupled to the PCB 710. The wireless transmission circuitry 750 can be configured to transmit using a local wireless protocol such as Bluetooth, a local wireless network such as WiFi, a wide area wireless network such as a cellular network, or another wireless transmission network or protocol.

The housing 210 can include a port 720 that has an opening 722 connected to the channel(s) 330 in the shaft 26. The port(s) 720 can be configured to connect to a tube or syringe for example using a Luer-lock fitting 724. The Luer-lock fitting 724 is shown as a female fitting but can be a male fitting in another embodiment. The Luer-lock fitting 724 can provide a leakproof connection with a standard male-taper fitting of a syringe tips and/or other fluid-transfer devices.

The opening 722 can be used to insert a guidewire, a tool, an acoustic enhancer (e.g., engineered microbubbles), fluids, and/or a therapeutic substance through the channel(s) 430 and out the opening 330 in the tip 30 (FIG. 3), for example to be placed at or near a target volume 40.

The housing 210 can include pins 730 to releasably attach the front (not illustrated) and the back 740 of the housing 210. The internal space 742 of the housing 210 can be potted to provide a fluid seal (e.g., for liquids and/or gasses), as well as to improve mechanical and electrical robustness of the final assembly.

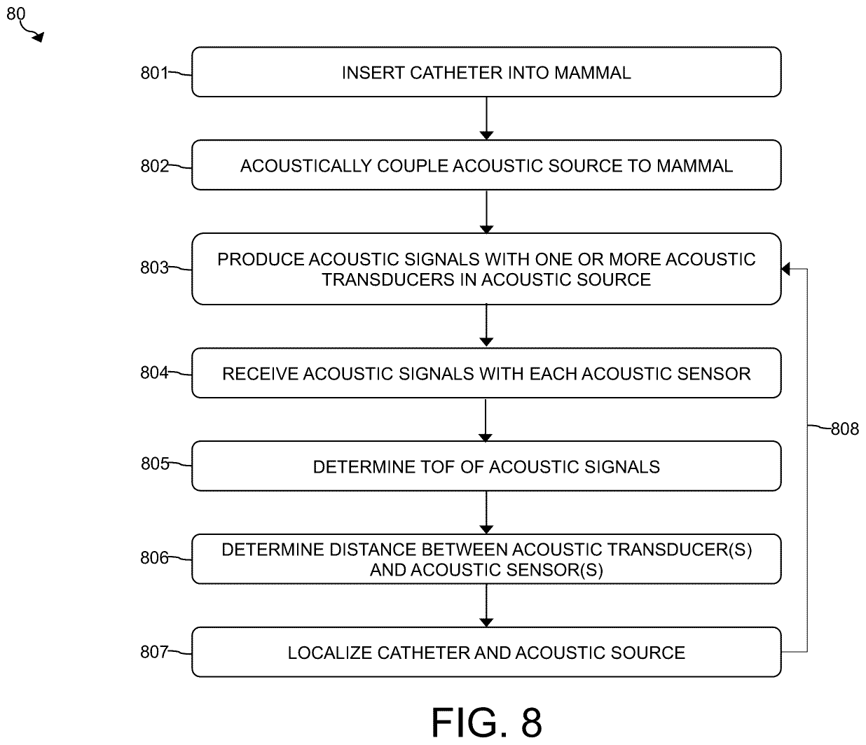

FIG. 8 is a flow chart of a method 80 for localizing an acoustic source 12 and a catheter 14 with respect to each other.

In step 801, the catheter 14 is inserted into a natural or surgical opening 32 in a mammal.

In step 802, the acoustic source 12 is acoustically coupled to a mammal 22 such as a human. The acoustic source 12 can be placed directly on the mammal 22 (e.g., on the skin 20 of the mammal 22) to acoustically couple the acoustic source 12 to the mammal 22. Alternatively, an acoustically transmitting media 24, such as water, a water cushion, an acoustic coupling oil, and/or an acoustic coupling gel, can be disposed between (e.g., in direct physical contact with) the acoustic source 12 and the skin 20 to improve acoustic transmission and acoustic coupling. The acoustic source 12 can be acoustically coupled at a position on the mammal 22 that corresponds to a target location of the introduced catheter 14.

In step 803, acoustic signals 34 are produced with one or more acoustic transducers 18 in the acoustic source 12. When the acoustic signals 34 are produced with multiple (e.g., two or more) acoustic transducers 18, the acoustic signals 34 are sequentially produced with each acoustic transducer 18.

The acoustic source 12 can include an array or another configuration of acoustic transducers 18. The array can include one row (e.g., a linear array) or two or more rows of acoustic transducers 18. In some embodiments, one of the acoustic transducers 18 can be positionally offset with respect to a linear array to break the symmetry of the acoustic transducers 18.

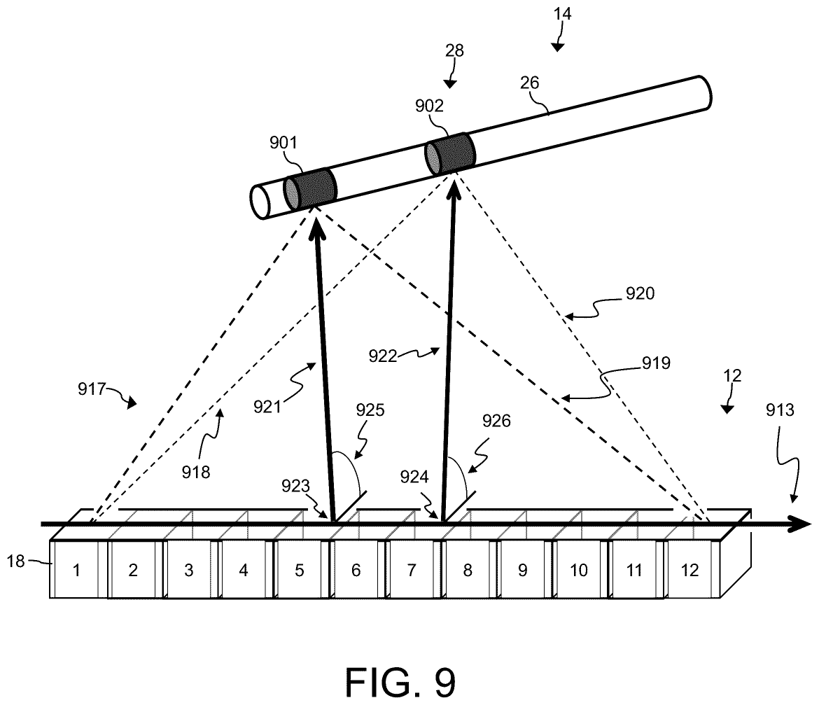

FIG. 9 illustrates an example linear array of acoustic transducers 18 in an acoustic source 12 and an example catheter 14, which is simplified as a cylinder for illustration purposes only. The acoustic signals 34 are sequentially produced by the first and last (numbers 1 and 12) acoustic elements. In other embodiments, the acoustic signals 34 can be sequentially produced by other and/or additional acoustic transducers 18. In other embodiments, the acoustic signals 34 are only produced by one acoustic transducer 18.

In step 804, the acoustic signals 34 are received by the acoustic sensor(s) 28 on or in the catheter 14. For example, in FIG. 9 the acoustic signals 34 are received by a first acoustic sensor 901 and a second acoustic sensor 902. When the acoustic signals 34 are sequentially produced by multiple acoustic transducers 18, the acoustic signals 34 are sequentially received by the first and second acoustic sensors 901, 902 from each acoustic transducer 18.

In step 805, the ToF(s) of the acoustic signals 34 transmitted between the acoustic transducer(s) 18 and the acoustic sensor(s) 28 is/are determined. The ToF(s) can be determined by a detector 42, such as a computer, that is electrically coupled to, in electrical communication, and/or in electromagnetic (e.g., wireless) communication with the acoustic sensor(s) 28. The detector 42 can be electrically coupled to, in electrical communication, and/or in electromagnetic (e.g., wireless) communication with a controller 25 and/or the source 12. The information from the detector 42 can be transmitted to a processing device and/or a display device.

The ToF can be determined as the arrival time of acoustic signal 34 to the acoustic sensor(s) 28 relative to a trigger signal provided by the controller 25. Alternatively, the ToF can be determined by measuring the time delay between the emitted and received signals. Alternatively, the ToF can be determined from the receiver signal alone because the acoustic sensor(s) 28 typically detects both the acoustic signal and EMI induced during the emission of the acoustic signals 34 by the emitting acoustic transducer(s) 18, such as a treatment head. The delay between the EMI and the detected acoustic signal at the acoustic sensor(s) 28 can be used to determine the ToF.

The delay between the emitted and received signals can be measured by finding the maximum of cross-correlation function of the emitted and received signals. Alternatively, the delay can be determined as a time difference between some characteristic features detectable in the emitted and received signals. The characteristic features can include a maximum amplitude or a certain threshold level, for example, in a range of about 10% to about 50% of the maximum amplitude. The characteristic features can also include signal peaks, valleys, and/or zero-crossings. These features can be used alone or in combination using sequences at one or plurality of frequencies. Furthermore, characteristic features can be created in acoustic waves using, for example, amplitude, phase, and/or frequency modulation. Some combination of these techniques may be used to improve the accuracy of the measurement of the delay time.

In yet another embodiment, the location of the sensor can be determined by considering the difference in arrival time from different transducer elements. The difference lies on a hyperbola with foci at the transducer locations. Different pairs of transducers can provide different hyperbolas. The intersection of the hyperbolas gives the location of the sensor. The mathematical formulation would be similar to that used in hyperbolic navigation.

In step 806, the distance between each acoustic sensor(s) 28 and each acoustic transducer(s) 18 is/are determined. The distance can be determined by multiplying the respective ToF by the speed of sound in soft tissue, which can be approximated as the speed of sound in water. The speed of sound in water at normal body temperature (37° C. or 98.6° F.), is 1524 m/s, about 2.6% greater than that at room temperature of 20° C.

In step 807, the catheter 14 and acoustic source 12 are localized with respect to each other. The localization resolution can vary depending on the number and/or configuration of acoustic transducers 18, including the number of transducer elements of the acoustic transducer(s) 18, that produce the acoustic signals 34 and/or the number of acoustic sensors 28. It is noted that the localization occurs without imaging, including acoustic (e.g., ultrasound) imaging. The relative positions of the acoustic source 12 and the catheter 14 can be displayed on a display screen 44 (FIG. 1) either wirelessly connected or in electrical communication with the detector 42.

In some embodiments, steps 803-807 can be repeated 808 such as during a medical procedure. Additionally or alternatively, the catheter 14 can be used to monitor the magnitude and/or other properties of therapeutic ultrasound (or other acoustic energy) produced by the acoustic source 12. The catheter 14 can also be used to introduce a guidewire, a tool, an acoustic enhancer (e.g., engineered microbubbles), fluids, and/or a therapeutic substance. Additionally or alternatively, steps 803-807 can be repeated 808 until the localization (e.g., roll, slide, and depth) are at a target value or within a target range. After the catheter 14 and acoustic source 12 are localized at a target value or within a target range, the position of the acoustic source 12 can be fixed or locked for example using a positioning accessory.

An example localization method is illustrated in FIG. 9. A reference axis 913 is positioned along the surface of the example linear array of acoustic transducers 18 of the acoustic source 12. Lines 921, 922 represent the radial distances, along respective radial lines, from the reference axis 913 to the first and second acoustic sensors 901, 902 on the catheter 14. Lines 921, 922 are orthogonal to the reference axis 913 and are oriented at respective angles 925 and 926 which can be the same or different from each other depending on the orientation of the catheter 14. Angles 925, 926 represent the angular coordinates of the first and second acoustic sensors 901, 902 in a cylindrical coordinate system with the longitudinal axis 913.

Measurements of ToF of the acoustic signals 34 from the acoustic transducers 18 to the first and second acoustic sensors 901, 902 allows one to determine the distance between the first and second acoustic sensors 901, 902 and acoustic transducers 18 (e.g., reference axis 913). Any pair of acoustic transducers 18 can be used to determine the following two coordinates of the first and second acoustic sensors 901, 902: the axial coordinates 923, 924 along the reference axis 913 and the radial distances 921, 922, respectively, from the reference axis 913. For example, distances from acoustic elements 1 and 12 of transducer 18 to the first and second acoustic sensors 901, 902 are shown with dashed lines 917-920. Considering the triangle formed by lines 917, 919, and a line (e.g., a segment of line 913) connecting the acoustic elements 1 and 12 of transducer 18, one can find the radial distance 921 between the first acoustic sensor 901 and the reference axis 913. Likewise, considering the triangle formed by lines 918, 920, and the line connecting the acoustic elements 1 and 12 of transducer 18, one can find the radial distance 922 between the second acoustic sensor 902 and the reference axis 913.

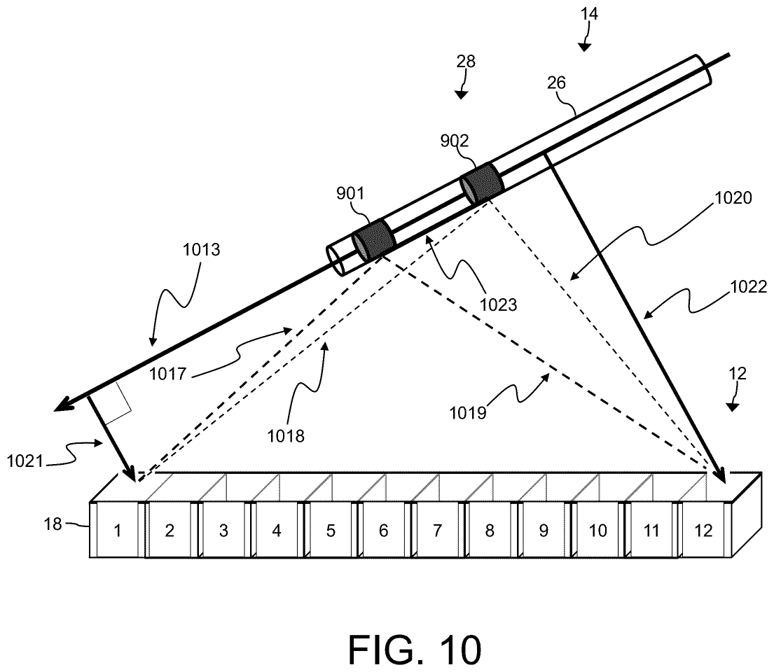

FIG. 10 illustrates an example localization method according to another embodiment. FIG. 10 represents a system of cylindrical coordinates with a reference axis 1013 passing along or through the catheter 14, where the catheter 14 remains still while the acoustic transducer 18 is movable. Lines 1021, 1022 represent the radial distances, along respective radial lines, from the reference axis 1013 to acoustic elements 1 and 12 of transducer 18, respectively. The distances from acoustic elements 1 and 12 of transducer 18 to the first and second acoustic sensors 901, 902 are shown with dashed lines 1017-1020. The radial distance 1021 from the reference axis 1013 to acoustic element 1 can be determined considering the triangle formed by lines 1017, 1018, and a line 1023 (e.g., a segment of reference axis 1013) that connects the first and second acoustic sensors 901, 902. Likewise, the radial distance 1022 from the reference axis 1013 to acoustic element 12 can be determined considering the triangle formed by lines 1019, 1020, and 1023. Lines 1021 and 1022 are generally skewed lines. That is, the triangle formed by lines 1017, 1018, and 1023 is not necessarily in the same plane with the triangle formed by lines 1019, 1020, and 1023.

One potential issue with the example localization methods illustrated in FIGS. 9 and 10 is that the symmetry of the linear array of acoustic transducers 18 may make it difficult to resolve all three coordinates of the respective position of each acoustic sensor 901, 902. All elements of a linear-array transducer are arranged along one line. The symmetry of the linear-array arrangement can pose a problem for a full three-dimensional position resolution. This problem is illustrated, for example with reference to in FIG. 9. Measurements of the distances between each acoustic sensors 901, 902 and any pair of acoustic transducers 18 allow determination of only two independent coordinates. In a cylindrical coordinate system, these coordinates are the position along the reference (or longitudinal) axis 913 and the radial distance (e.g., along lines 921, 922) from the reference axis 913. For the first acoustic sensor 901, for example, the radial distance from the reference axis 913 is shown with 921. The dashed lines 917, 919 connect the first acoustic sensor 901 with the acoustic elements 1 and 12, respectively, and represent the respective distance between the first acoustic sensor 901 and the acoustic elements 1 and 12. Consideration of any other colinear elements (for example, any of acoustic elements 2-11) does not resolve the angular coordinate 925—the third independent coordinate of the cylindrical system-because all the transducer elements are on the same line and in the same plane as lines 917 and 919. To determine the angle 925, one needs additional information.

In an embodiment, the problem associated with the symmetry of a linear-array arrangement can be resolved using either a 2D or 3D arrangement of the acoustic sensors 28 (e.g., acoustic sensors 901, 902) and/or of the acoustic transducers 18. For example, the catheter 14 can have three or more noncolinear acoustic sensors 28. Alternatively, the acoustic transducers 18 can be arranged either on a 2D surface or a 3D geometry. Note that to resolve the symmetry problem, it is sufficient to break the symmetry of a linear array by either separating, splitting, or adding one or more elements in the direction normal to the longitudinal axis (reference axis 913 in FIG. 9) of the linear array. For example, the acoustic transducers 18 can be arranged as a two-dimensional array or a three-dimensional array. Alternatively, one or more additional acoustic transducers 18 can be added to in a noncolinear manner to the linear array.

Another approach for localization is to employ a known distribution of acoustic field produced by a given transducer. Matching the known acoustic distribution with the measurements of the acoustic signal by the catheter sensors (e.g., acoustic sensors 28) allows one to determine the position of the catheter in the acoustic field. One example of this general approach is to use a known angular dependence of acoustic signal to assess the third independent coordinate. In FIG. 9, for example, such third coordinate is the angular coordinate 925, 926 of a cylindrical coordinate system. As illustrated in FIG. 9, the acoustic transducers 18 of the linear array have a finite thickness in the direction perpendicular to the reference axis 913. Therefore, these elements can generate acoustic fields with specific patterns characterized by an angular dependence. The angular dependence can affect both acoustic amplitude and/or the duration of the acoustic signal. Matching the angular dependence with acoustic signal measurements can allow one to determine the angular coordinates 925, 926.

Another approach for localization is to separate the acoustic sensors 28 (e.g., acoustic sensors 901, 902) mounted on the catheter 14 by a known distance. This distance provides an additional restriction that reduces ambiguity of localization. Consider, for example, a catheter with two sensors and a linear-array transducer shown in FIG. 9. The positions of the first and second acoustic sensors 901, 902 are described by axial coordinates 923, 924, radial distances 921, 922, and angles 925, 926, respectively. As discussed above, ToF measurements allow one to determine the axial coordinates 923, 924 and radial distances 921, 922, but not the angles 925, 926 due to the symmetry of the linear-array transducer. Use of a known distance between the first and second acoustic sensors 901, 902 allows one to assess the angle between the reference axis 913 and the direction of the catheter to determine the angles 925, 926.

It is noted that when the catheter 14 includes only one acoustic sensor 28, the direction of the catheter 14 is difficult to determine. The distance between the catheter 14 and the acoustic source 18 can be determined. Referring to FIG. 9 and assuming that the catheter 14 includes only the first acoustic sensor 901, the radial distance along radius line 921 and the axial coordinate 923 of the first acoustic sensor 901 (e.g., of the radius line 921) can be determined when at least two acoustic transducers 18 produce the acoustic signals 34. The angle 925 of the first acoustic sensor 901 (e.g., of the radius line 921) can be determined using, for example, the angular dependence of the acoustic signal on orientation of the acoustic transducer 18, as discussed above.

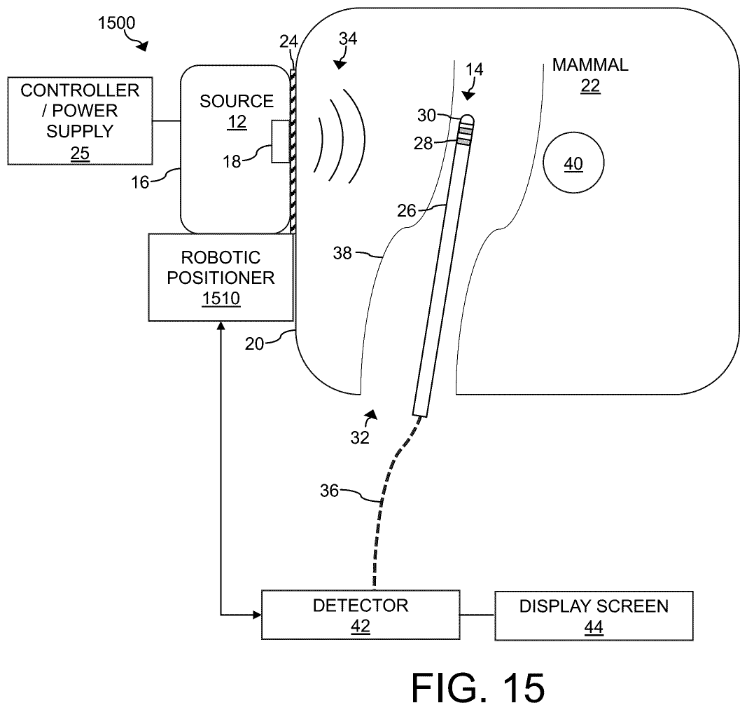

In some embodiments, the detector 42 can use the localization data to produce output signals that cause a robotic positioner 1510 (FIG. 15) to position the acoustic source 12 relative to the catheter, for example to align the acoustic source 12 with respect to the catheter 14. The robotic positioner 1510 and the detector 42 can communicate wirelessly or electrically or both.

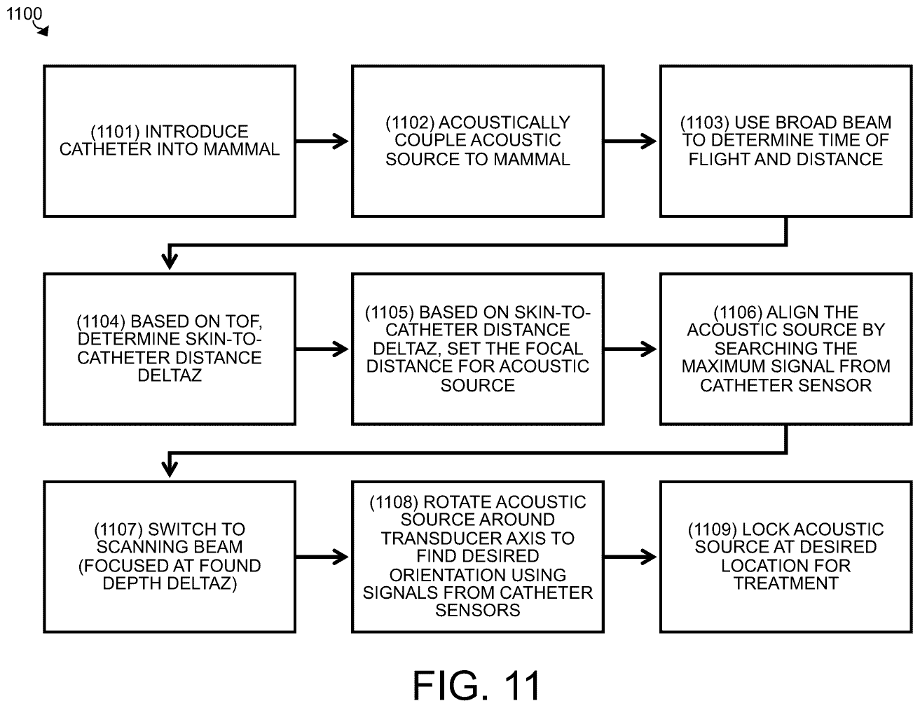

FIG. 11 is a flow chart of a method 1100 for localizing an acoustic source 12 and a catheter 14 with respect to each other according to another embodiment.

In step 1101, the catheter 14 is inserted into a natural or surgical opening 32 in a mammal.

In step 1102, the acoustic source 12 is acoustically coupled to a mammal 22 such as a human. The acoustic source 12 can be placed directly on the mammal 22 (e.g., on the skin 20 of the mammal 22) to acoustically couple the acoustic source 12 to the mammal 22. Alternatively, an acoustically transmitting media 24, such as water, a water cushion, an acoustic coupling oil, and/or an acoustic coupling gel, can be disposed between (e.g., in direct physical contact with) the acoustic source 12 and the skin 20 to improve acoustic transmission and acoustic coupling.

In step 1103, a broad beam of transcutaneous ultrasound is produced to determine the skin-to-catheter distance by measuring the ToF between the acoustic transducer(s) 18 and the acoustic sensor(s) 28. The ToF is used to determine the distance between the acoustic transducer(s) 18 and the acoustic sensor(s) 28 in step 1104. In step 1105, based on the measured distance (e.g., skin-to-catheter distance), the focal distance for the acoustic beam produced by the acoustic transducer(s) 18 is set.

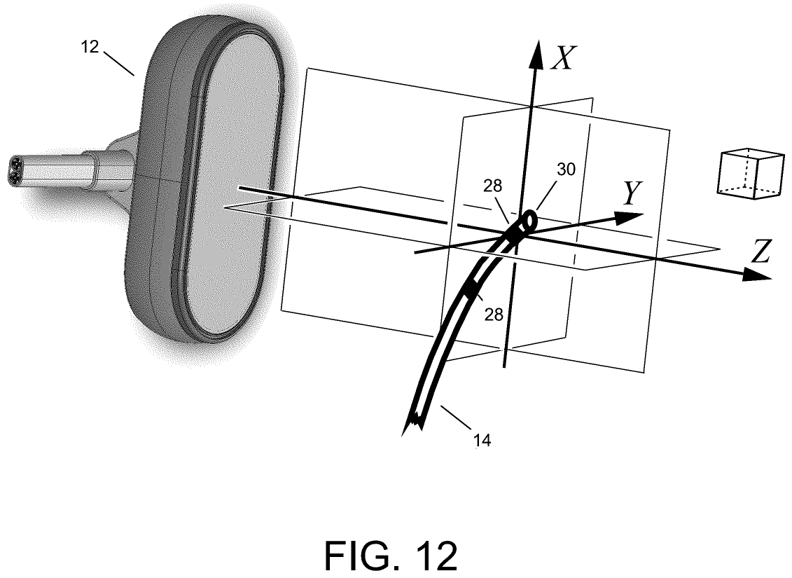

In step 1106, the acoustic transducer(s) 18 produce acoustic signals 34 that are focused at the focal distance determined in step 1104. When the acoustic source 12 only includes one acoustic transducer 18, the acoustic transducer 18 can include adjustable acoustic lenses and/or mechanical means to vary the focal distance. The acoustic source 12 is aligned with the catheter 14 by searching for a first maximum-amplitude signal measured by the acoustic sensor(s) 28 as the acoustic source 12 is moved or translated parallel to the Y-axis of the XYZ-coordinate system shown in FIG. 12. The first maximum-amplitude signal corresponds to a first localization, such as a first coordinate (e.g., with respect to the Y-axis) and a corresponding position of the acoustic source 12 (e.g., with respect to the Y-axis) on the mammal 22. In step 1107, once the acoustic source 12 is aligned with the acoustic sensor(s) 28, the acoustic field on the acoustic source 12 is switched to a sweeping focused beam while the acoustic source 12 remains at the position along the Y-axis that corresponds to the first maximum-amplitude signal. The focused beam can be swept by moving the acoustic source 12 parallel to the Y-axis and/or by varying the focal position electronically (e.g., by varying relative phases of the acoustic transducers 18 in a phased array). This will sweep the acoustic beam along or parallel to the X-axis of the XYZ-coordinate system shown in FIG. 12.

In step 1108, the acoustic source 12 is rotated about the transducer axis to find the orientation when signals on the acoustic sensor(s) 28 are maximized, indicating good alignment of the treatment head to the catheter in the ureter. In step 1109, the aligned acoustic source 12 is locked in place (e.g., at the position corresponding to the first maximum-amplitude signal) to perform a treatment procedure. The acoustic source 12 can be locked in place with a mechanical apparatus. It is noted that the localization occurs without imaging, including acoustic (e.g., ultrasound imaging) imaging.

FIG. 12 shows an example Cartesian coordinate system with three orthogonal coordinates X, Y, and Z. The X-axis is directed along the longitudinal axis of the acoustic source 12 (e.g., treatment head). The Z-axis is directed along the axis of symmetry of the acoustic source 12. The distal end 202 (FIG. 2) of the catheter 14 is located at distance Z from the acoustic source 12.

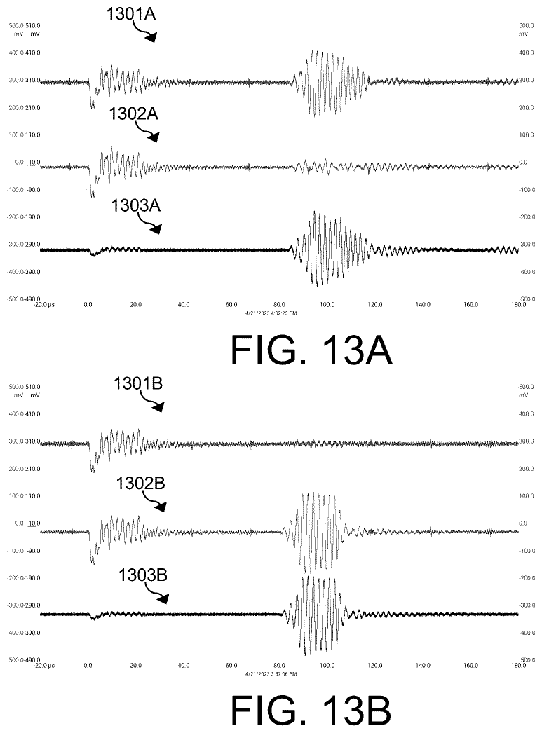

FIGS. 13A and 13B illustrate example traces 1301A, 1301B, 1302A, 1302B of two acoustic sensors 28 on a catheter 14 and their respective differential signals 1303A, 1303B demonstrating an alignment procedure for an acoustic source 12 to a catheter 14. One acoustic sensor 28 was mounted at about 3 cm from the catheter tip (traces 1301A, 1301B), the other near the tip (traces 1302A, 1302B). The catheter 14 was positioned along the X-axis of the acoustic source 12 (FIG. 12) at about 12 cm distance (Z-axis) from the acoustic source 12, corresponding to a ToF of about 80 μs. The acoustic source 12 emitted 10-cycle tone bursts at a center frequency of 450 kHz. The acoustic signals received by the acoustic sensors 28 were seen in a time window from 80-120 μs. In FIG. 13A, trace 1301A showed a greater amplitude of acoustic signal than that of trace 1302A detected by the transducer at the catheter tip. The displacement of the treatment head by 3 cm along the X-axis decreased the trace 1301B and increased the trace 1302B, indicating alignment of the treatment head 12 to the tip of catheter 14. The signal traces 1303A, 1303B show a differential signal, calculated by taking a difference between signals received by the two sensors 1301 and 1302, demonstrating a method to reduce EMI induced during emission of the acoustic source 12.

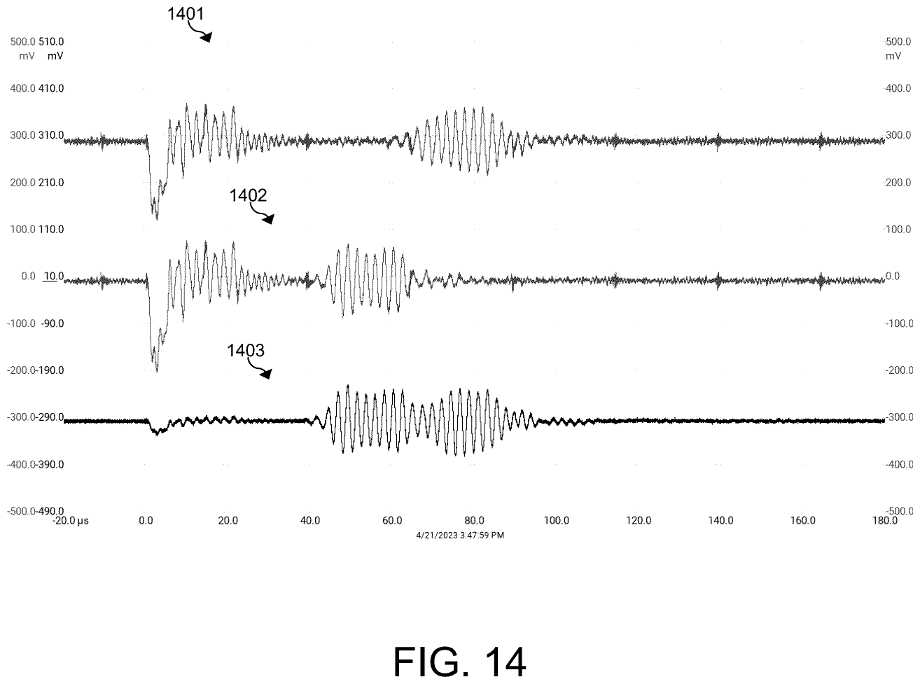

FIG. 14 shows signals from acoustic sensors 28 (traces 1401, 1402) mounted approximately 3 cm apart on a catheter 14 oriented along the acoustic axis Z of the acoustic source 12. The travel time of the acoustic signals over 3 cm distance was 20 μs. This time delay was observed between the traces 1401, 1402 indicating that the Z-axis of the treatment head was oriented such that the acoustic axis (Z-axis, FIG. 12) was along the length of the catheter 14. Furthermore, a shorter arrival time on the acoustic sensor 28 positioned closer to the tip 30 of the catheter 14 (trace 1402) compared to that of the acoustic sensor 28 positioned further from the tip 30 (trace 1401) indicates that the tip 30 of the catheter 14 was oriented toward the acoustic source 12. This orientation of the catheter 14 was confirmed visually, demonstrating the proposed alignment method. Trace 1403 represents a differential signal, demonstrating a method to reduce EMI induced simultaneously on both acoustic sensors 28 during the emission of the treatment head (about 0-20 μs). The acoustic source 12 emitted a 10-cycle tone burst with a center frequency of 450 kHz. The acoustic signals received by the acoustic sensors 28 were seen in time window from 40-100 μs, corresponding to the ToF for acoustic wave from the acoustic source 12 to the acoustic sensors 28.

A catheter 14 with acoustic sensors 28 can be used for guidance in medical procedures. The catheter 14 can be inserted either through a minute incision through the skin or through a natural orifice in the human body. Such applications include, for example, a biopsy of various organs or for percutaneous access in the kidney. For percutaneous access, the catheter 14 can be inserted through the ureter and can have a pre-set shape that will ensure a certain position of the catheter 14 in the kidney. For example, the catheter 14 can be designed such that it prefers a certain shape such that it tends to be positioned in the low pole of the kidney. The ultrasound source 12 can include a guiding fixture to guide a needle which can be used for the medical procedure such as for a biopsy or a percutaneous nephrolithotomy (PCNL). The guiding fixture can be adjustable so as to be oriented at the localized position of the catheter 14. The system can determine the depth for needle insertion, which can be the same as the measured depth or distance between the acoustic source 12 and the catheter 14, and can make sure that the needle avoids blood vessels, which is a typical concern and/or complication during the percutaneous access. An apparent advantage of all the potential applications of the catheter with acoustic sensors is avoiding or minimizing the use of harmful X-ray radiation that is currently used for guidance in these procedures.

Another unique advantage of a catheter 14 with acoustic sensors 28 is that this device provides absolute measurements of acoustic pressure in the treatment zone. This is important since the acoustic transmission is typically affected by various factors, such as, for example, mismatch of acoustic impedances at various interfaces, low acoustic transmission through ribs and bones, difference in attenuation of acoustic waves in different types of tissue, and presence of air or gas pockets along the acoustic path. Because of these factors, the actual acoustic pressure delivered to the treatment zone is typically unknown. This weakness can be addressed by using a catheter 14 with acoustic sensors 28.

FIG. 15 is a block diagram of a system 1500 for acoustically localizing an acoustic source 12 with respect to a catheter 14 according to another embodiment. System 1500 is the same as system 10 (FIG. 1) except that in system 1500 a robotic positioner 1510 is electromechanically coupled to the acoustic source 12. The detector 42 can send output control signals to the robotic positioner 1510 that cause the robotic positioner 1510 to position the acoustic source 12 in response to a localization of the acoustic source 12 and the catheter 14 relative to each other. For example, the robotic positioner 1510 can align the acoustic source 12 with respect to the catheter 14. The detector 42 and the robotic positioner 1510 can be connected electrically and/or wirelessly.

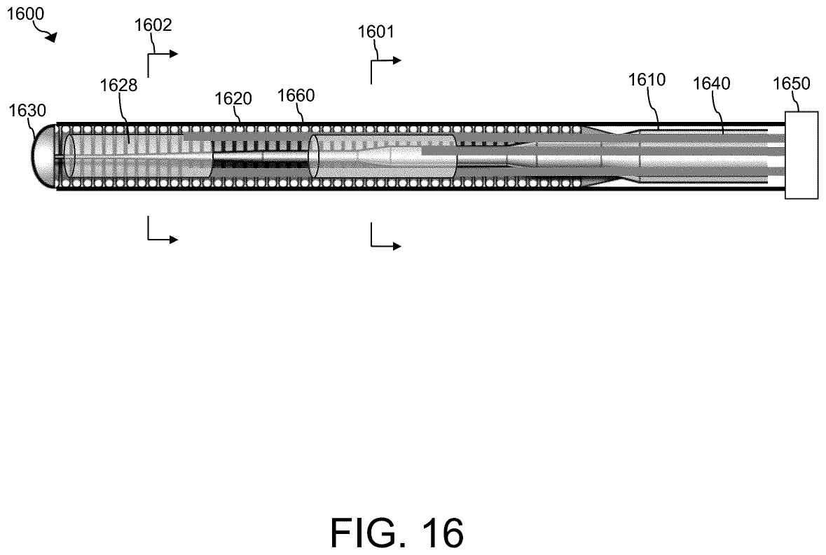

FIG. 16 is a partially transparent side view of a guidewire 1600 according to an embodiment. The guidewire 1600 includes a core 1610, an optional coil 1620, and one or more acoustic sensors 1628. The coil 1620 can be removed in some embodiments. The core 1610 and the coil 1620 are coaxial and extend from a proximal end to a distal end of the guidewire 1600. The acoustics sensor(s) 1628 can be located at predetermined position(s) from the distal end of the guidewire 1600 and/or from a tip 1630 at the distal end of the catheter 1600.

The acoustic sensor(s) 1628 is/are located between the core 1610 and the coil 1620 and are disposed about at least a portion of the core 1610. The acoustic sensor(s) 1628 are electrically connected to leads and/or wires 1640. The leads and/or wires 1640 can be electrically connected to one or more electrical connection points in a housing 1650 attached to a proximal end of the guidewire 1600. It is noted that the guidewire 1600 is not shown at scale and in practice the housing 1650 would be much further away from the tip 1630 and the acoustic sensor(s) 1628 than as illustrated.

The leads and/or wires 1640 can be disposed or embedded in a protective coating 1660 that covers the external surface of the guidewire 1600, such that the leads and/or wires 1640 extend parallel to the core 1610. The coil 1620 can be disposed or embedded in the protective coating 1660. In another embodiment, the core 1610 can be hollow and the leads and/or wires 1640 can pass through a channel in the core 1610.

The acoustic sensor(s) 1628 can be the same as the acoustic sensors 28. Additionally or alternatively, the housing 1650 can be the same as the housing 210.

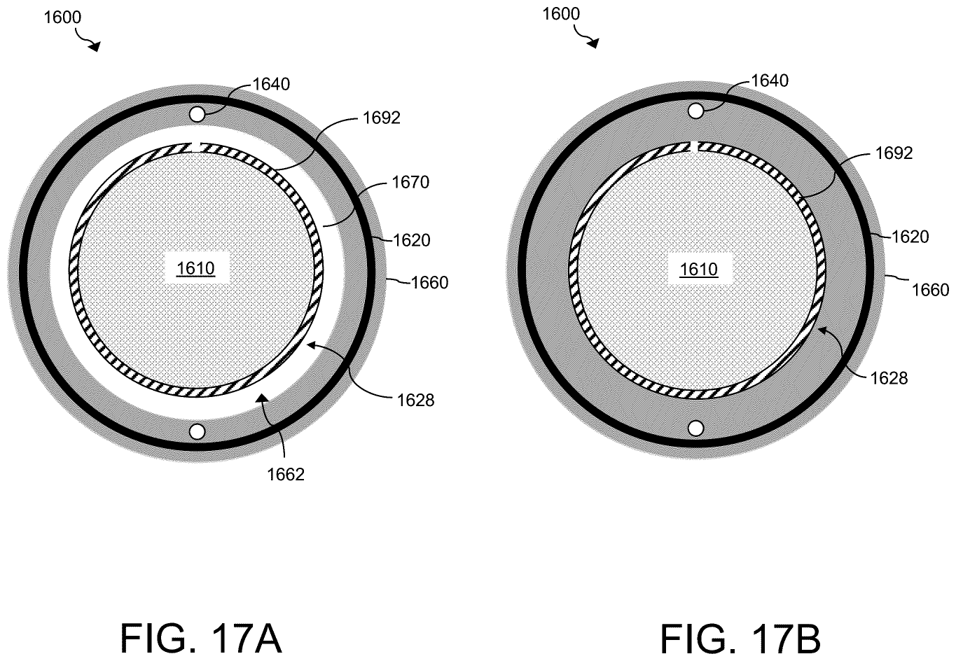

FIG. 17A is a cross section of the guidewire 1600 through plane 1601 in FIG. 16 according to an embodiment. The acoustic sensor 1628 includes a piezoelectric polymer film 1692 that is disposed about at least a portion of the circumference of the core 1610. The piezoelectric polymer film 1692 can be the same as the piezoelectric polymer film 392. In some embodiments, the acoustic sensor 1628 can include two piezoelectric polymer films (e.g., as illustrated in FIG. 6D).

To reduce or eliminate air gaps between the protective coating 1660 and the sensor 1628 (e.g., piezoelectric polymer film 1692), a gap 1662 between the protective coating 1660 and the sensor 1628 can be filled with one or more materials 1670 that have an acoustic impedance that matches (e.g., within about 20%) the impedance of bodily fluids and/or tissue, which have an acoustic impedance similar to that of water. The material(s) 1670 can be the same as the material(s) 600. In another embodiment, air gaps can be removed or reduced by the increasing the diameter of the core 1610 and/or by increasing the thickness of the protective coating 1660 so that the piezoelectric polymer film 1692 is in direct physical contact with the protective coating 1660 as well as with the core 1610, as illustrated in FIG. 17B.

A cross section of the guidewire 1600 through plane 1602 in FIG. 16 can be the same as the cross section illustrated in FIG. 17A or the cross section illustrated in FIG. 17B though the diameter of the core 1610 may be smaller in the cross section through plane 1602 than in the cross section through plane 1601.

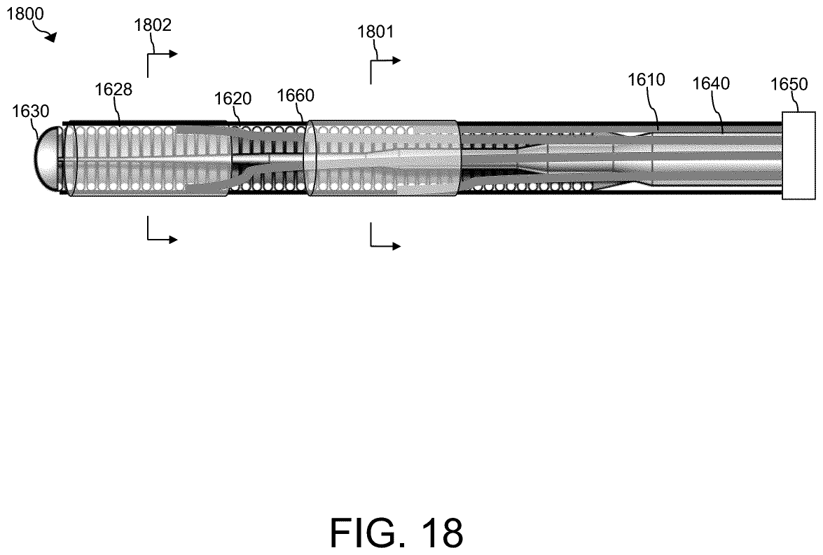

FIG. 18 is a partially transparent side view of a guidewire 1800 according to another embodiment. The guidewire 1800 is the same as the guidewire 1600 except that in the guidewire 1800 the acoustic sensor(s) 1628 is/are disposed on or in the protective coating 1660.

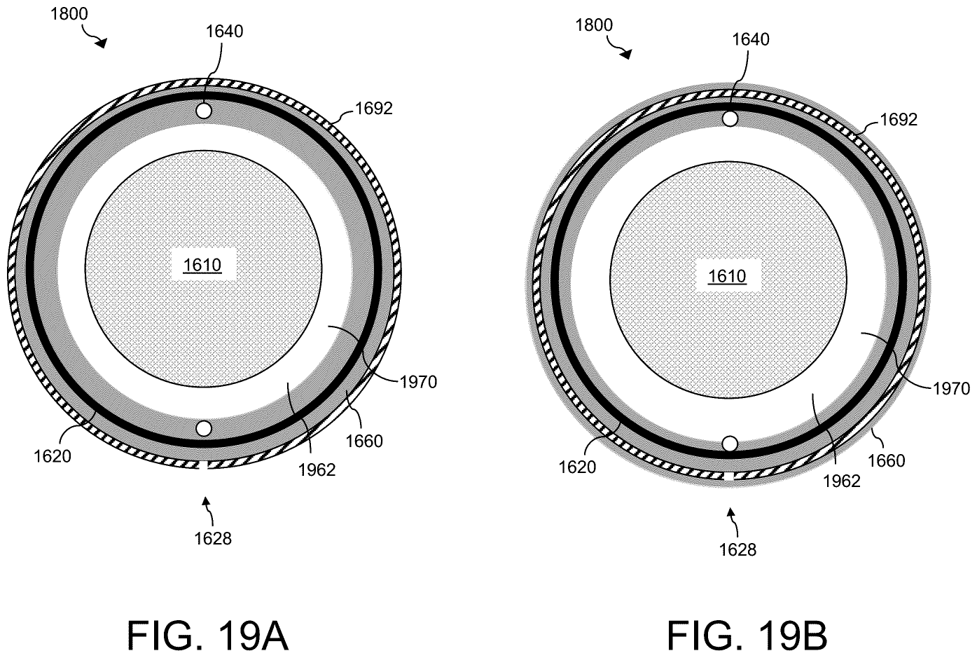

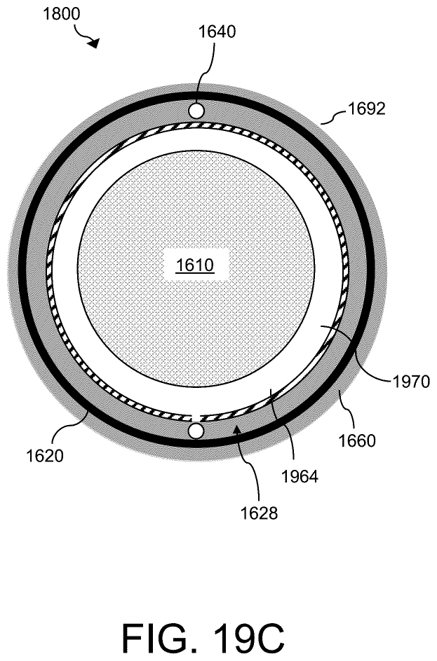

FIG. 19A is a cross section of the guidewire 1800 through plane 1801 in FIG. 18 according to an embodiment. The acoustic sensor 1628 includes a piezoelectric polymer film 1692 that is disposed on the external surface of the protective coating 1660. The piezoelectric polymer film is disposed about at least a portion of the circumference of the protective coating 1660. A gap 1962 between the protective coating 1660 and the core 1610 can be filled with one or more materials 1970 having a high acoustic impedance and/or that can provide an acoustically rigid interface to increase acoustic pressure at the acoustic sensor 1628. In some embodiments, the material(s) 1970 can be configured to absorb acoustic waves. Partial or complete absorption of acoustic waves (such as by an epoxy resin) can reduce or eliminate the reflection of acoustic waves, which may be desirable in some embodiments. The material(s) 1970 can be the same as the material(s) 610.

In some embodiments, the acoustic sensor 1628 can include two piezoelectric polymer films (e.g., as illustrated in FIG. 6D).

FIG. 19B is a cross section of the guidewire 1800 through plane 1801 in FIG. 18 according to another embodiment. The acoustic sensor 1628 includes a piezoelectric polymer film 1692 that is disposed or embedded in the protective coating 1660. The piezoelectric polymer film is disposed about at least a portion of the protective coating 1660. The gap 1962 can be filled with one or more materials 1970.

FIG. 19C is a cross section of the guidewire 1800 through plane 1801 in FIG. 18 according to another embodiment. The acoustic sensor 1628 includes a piezoelectric polymer film 1692 that is disposed on the internal surface of the protective coating 1660. The piezoelectric polymer film is disposed about at least a portion of the circumference of the inner diameter of the protective coating 1660. A gap 1964 between the piezoelectric polymer film 1692 and the core 1610 can be filled with one or more materials 1970.

A cross section of the guidewire 1800 through plane 1802 in FIG. 18 can be the same as any of the cross sections illustrated in FIG. 19A-C though the diameter of the core 1610 may be smaller in the cross section through plane 1602 than in the cross section through plane 1601.

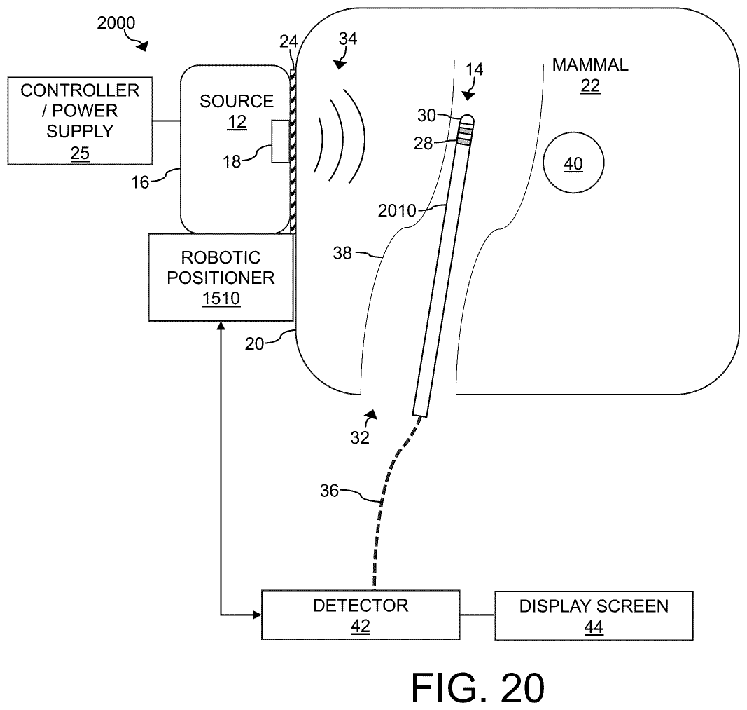

FIG. 20 is a block diagram of a system 2000 for acoustically localizing an acoustic source 12 with respect to a guidewire 2010 according to an embodiment. The system 2000 is the same as the system 10 and/or the system 1500 except that the catheter 14 in systems 10, 1500 is replaced with the guidewire 2010. The guidewire 2010 can be the same as the guidewire 1600 or the guidewire 1800.

An acoustic source 12 and a guidewire 2010 can be localized with respect to each other according to method 80 (FIG. 8) or according to method 1100 (FIG. 11) where the catheter 14 is replaced with a guidewire 2010.

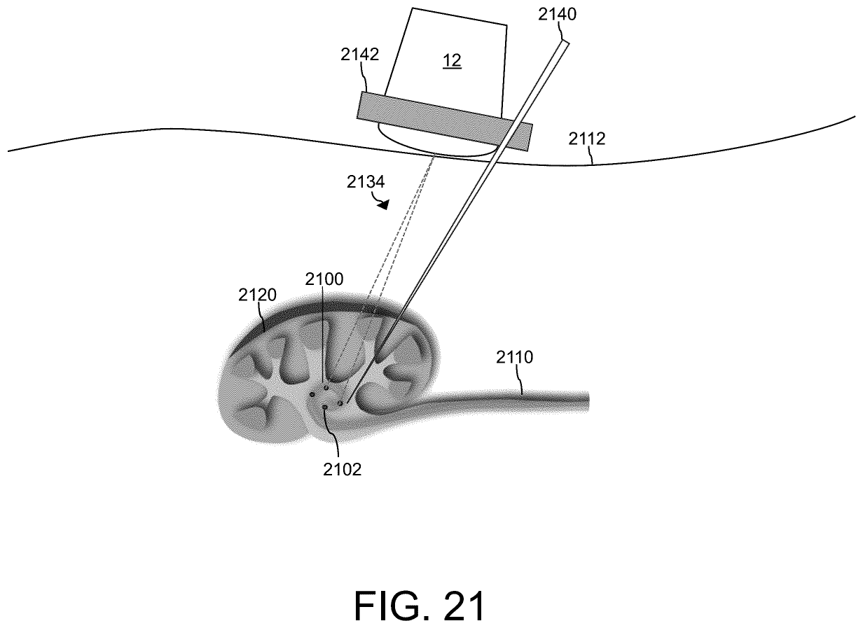

FIG. 21 shows an embodiment in which a medical device 2100 with multiple acoustic sensors 2102 is inserted through the ureter 2110 in the collecting system of the human kidney 2120. The medical device 2100 can be a catheter (e.g., catheter 14), a guidewire (e.g., guidewire 1600, 1800), or another medical device. The acoustic sensors 2102 can be the acoustic sensor(s) 28, the acoustic sensor(s) 1628, and/or the acoustic sensor(s) 1828.

When fully inserted, the medical device 2100 is configured to coil or uncoil, accepting a predetermined shape, e.g., forming a coil-like structure with a predetermined shape in the kidney 2120 or in another target location. The predetermined shape determines the relative position of the acoustic sensors 2102 to avoid the possible ambiguity of sensor position in the kidney 2120. An extracorporeal acoustic source 12, acoustically coupled to the patient's skin 2112, produces acoustic signal(s) 2134 which is received by the acoustic sensors 2102 to establish and/or determine the relative position of the acoustic source 12 and the acoustic sensors 2102 in the kidney 2120, for example using a detector 42. The acoustic signal(s) 2134 can be the same as the acoustic signals 34.

After the relative position of the acoustic source 12 and the acoustic sensors 2102 in the kidney 2120 is determined, a needle 2140 or similar medical device can now be inserted into the kidney 2120 (e.g., into the kidney calyx or a kidney wall), e.g., to perform a percutaneous access procedure, under the acoustic guidance of the acoustic source 12 and acoustic sensors 2102, as disclosed herein. The relative position and orientation (e.g., angle) of the needle 2140 is known relative to the acoustic source 12, for example using a needle guide or a needle bracket 2142, which can have an adjustable angle of insertion for the needle 2140. This approach is easy to perform and does not require special skills, such as a sonographic expert. This approach requires neither ultrasound imaging system nor harmful ionizing radiation imaging, such as X-ray imaging.

The invention should not be considered limited to the particular embodiments described above. Various modifications, equivalent processes, as well as numerous structures to which the invention may be applicable, will be readily apparent to those skilled in the art to which the invention is directed upon review of this disclosure. The above-described embodiments may be implemented in numerous ways. One or more aspects and embodiments involving the performance of processes or methods may utilize program instructions executable by a device (e.g., a computer, a processor, or other device) to perform, or control performance of, the processes or methods.

In this respect, various inventive concepts may be embodied as a non-transitory computer readable storage medium (or multiple non-transitory computer readable storage media) (e.g., a computer memory of any suitable type including transitory or non-transitory digital storage units, circuit configurations in Field Programmable Gate Arrays or other semiconductor devices, or other tangible computer storage medium) encoded with one or more programs that, when executed on one or more computers or other processors, perform methods that implement one or more of the various embodiments described above. When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the aspects described above. In some embodiments, computer readable media may be non-transitory media.

The terms “program,” “app,” and “software” are used herein in a generic sense to refer to any type of computer code or set of computer-executable instructions that may be employed to program a computer or other processor to implement various aspects as described above. Additionally, it should be appreciated that, according to one aspect, one or more computer programs that when executed perform methods of this application need not reside on a single computer or processor but may be distributed in a modular fashion among a number of different computers or processors to implement various aspects of this application.

Computer-executable instructions may be in many forms, such as program modules, executed by one or more computers or other devices. Generally, program modules include routines, programs, objects, components, data structures, etc. that performs particular tasks or implement particular abstract data types. The functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Thus, the disclosure and claims include new and novel improvements to existing methods and technologies, which were not previously known nor implemented to achieve the useful results described above. Users of the method and system will reap tangible benefits from the functions now made possible on account of the specific modifications described herein causing the effects in the system and its outputs to its users. It is expected that significantly improved operations can be achieved upon implementation of the claimed invention, using the technical components recited herein.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A catheter comprising:
a shaft;
a tip disposed at a distal end of the shaft;
at least one acoustic sensor disposed on or in the shaft, each acoustic sensor disposed at a respective distance from the distal end of the shaft; and
at least one electrical conductor disposed on or in the shaft, each electrical conductor electrically connecting a respective acoustic sensor to one or more electrical connection points in a housing attached to a proximal end of the shaft.

2. The catheter of claim 1, wherein each acoustic sensor comprises a piezoelectric polymer film disposed about at least a portion of a circumference of the shaft.

3. The catheter of claim 2, wherein the piezoelectric polymer film comprises polyvinylidene fluoride.

4. The catheter of claim 2, wherein:
the shaft includes an inner tube and an outer tube,
the respective piezoelectric polymer film is disposed about at least a portion of a circumference of the inner tube, and
the at least one electrical conductor is disposed between the inner and outer tubes.

5. The catheter of claim 4, wherein:
the inner tube is defined by a wall having an inner-wall thickness, and one or more regions of the wall have an increased thickness compared to the inner-wall thickness.

6. The catheter of claim 4, wherein:
the outer tube is defined by a wall having an outer-wall thickness, and one or more regions of the wall have an increased thickness compared to the outer-wall thickness.

7. The catheter of claim 4, wherein a spacer is disposed between the inner and outer tubes.

8. The catheter of claim 1, wherein the one or more electrical connection points is/are electrically coupled to a cable that extends through the housing.

9. The catheter of claim 8, wherein the one or more electrical connection points is/are formed on a printed circuit board disposed in the housing.

10. The catheter of claim 8, further comprising wireless communication circuitry electrically coupled to the one or more electrical connection points.

11. The catheter of claim 1, wherein the housing includes a port having a hole that is aligned with a central channel of the shaft.

12. The catheter of claim 1, wherein the at least one acoustic sensor includes first and second acoustic sensors, the first and second sensors separated by a predetermined distance.

13. A method for localizing an acoustic source and a medical device with respect to each other, the method comprising:
a. introducing the medical device into a mammal;
b. acoustically coupling the acoustic source to the mammal at a position that corresponds to a target location of the medical device, the acoustic source comprising a housing and a source transducer disposed in the housing;
c. producing acoustic signals with the source transducer;
d. receiving the acoustic signals with an acoustic sensor on or in the medical device, the acoustic sensor in electrical or wireless communication with a detector;
e. determining, with the detector, a time-of-flight (ToF) of the acoustic signals transmitted between the source transducer and the acoustic sensor;
f. determining, with the detector and using the ToF, a distance between the source transducer and the acoustic sensor; and
g. localizing, with the detector, the acoustic source and the medical device with respect to each other in real time based, at least in part, on the distance between the source transducer and the acoustic sensor.

14. The method of claim 13, wherein:
the acoustic sensor is a first acoustic sensor, the medical device includes at least a second acoustic sensor, and the method further comprises:
receiving the acoustic signals with the first and second acoustic sensors;
determining, with the detector, a first ToF of the acoustic signals transmitted between the source transducer and the first acoustic sensor;
determining, with the detector, a second ToF of the acoustic signals transmitted between the source transducer and the second acoustic sensor;
determining, with the detector and using the first ToF, a first distance between the source transducer and the first acoustic sensor;
determining, with the detector and using the second ToF, a second distance between the source transducer and the second acoustic sensor; and
localizing the acoustic source and the medical device with respect to each other based, at least in part, on the first and second distances.

15. The method of claim 13, wherein:
the medical device includes a plurality of acoustic sensors,
the acoustic source includes a plurality of source transducers, and
the method further comprises:
sequentially producing the acoustic signals with at least a first source transducer and a second source transducer of the plurality of source transducers;
receiving the acoustic signals with each acoustic sensor;
determining a respective ToF of the acoustic signals transmitted between (a) each of the at least the first source transducer and the second source transducer and (b) each acoustic sensor;
determining, using each ToF, respective distances between (a) each of the at least the first source transducer and the second source transducer and (b) each acoustic sensor; and
localizing the acoustic source and the medical device with respect to each other based, at least in part, on the respective distances.

16. The method of claim 13, wherein:
the position is a first position, and
the method further comprises after performing at least steps b-f while the acoustic source is located at the first position, moving the acoustic source to a second position and repeating steps b-f while the acoustic source is located at the second position so as to improve a resolution of a localization of the acoustic source and the medical device with respect to each other compared to when the localization is performed while the acoustic source is only located at the first position.

17. The method of claim 13, wherein:
the medical device comprises a catheter,
the catheter is introduced into an organ, and
the method further comprises:
introducing, with the catheter, an acoustic enhancer proximal to a calcification;
applying acoustic energy with the acoustic source; and
producing cavitation with the acoustic energy and the acoustic enhancer to disintegrate at least a portion of calcification.

18. The method of claim 13, further comprising adjusting a position of the acoustic source, with a robotic positioner in communication with the detector, according to a localization of the acoustic source and the medical device with respect to each other.

19. The method of claim 13, further comprising displaying relative positions of the acoustic source and the medical device on a display screen on or in electrical communication with the detector.

20. The method of claim 13, wherein the medical device comprises a catheter or a guidewire.

21. A method for localizing an acoustic source and a catheter with respect to each other, the method comprising:
a. introducing the catheter into a mammal, the catheter comprising:
a shaft and a tip disposed at a distal end of the shaft; and
at least one acoustic sensor disposed on or in the shaft, each acoustic sensor dispose at a respective distance from the distal end of the shaft;
b. acoustically coupling the acoustic source to the mammal at a position that corresponds to a target location of the catheter, the acoustic source comprising a housing and a plurality of source transducers disposed in the housing;
c. producing a broad beam of acoustic energy with the acoustic source;
d. determining, with a detector in electrical or wireless communication with the at least one acoustic sensor, a measured distance between the source transducers and the at least one acoustic sensor, the measured distance based, at least in part, on a time-of-flight (ToF) of the acoustic signals transmitted between the source transducers and each acoustic sensor;
e. setting a focal distance for the source transducers corresponding to the measured distance;
f. producing a focused beam of acoustic energy with the acoustic source while moving the acoustic source parallel to a first axis that is orthogonal to an acoustic axis of the acoustic transducers, the focused beam focused at the focal distance;
g. monitoring, with the detector, output signals of the at least one acoustic sensor to determine a first maximum amplitude signal while the focused beam is produced, the first maximum amplitude representing a first localization with respect to the first axis;
h. after step g, sweeping the focused beam with respect to a second axis that is orthogonal to an acoustic axis of the acoustic transducers, the ultrasound source located at a position corresponding to the first maximum amplitude signal;
i. monitoring, with the detector, the output signals of the at least one acoustic sensor to determine a second maximum amplitude signal while the focused beam is swept, the second maximum amplitude representing a second localization with respect to the second axis;
j. after step h, rotating the focused beam with respect to the acoustic axis; and
k. monitoring, with the detector, the output signals of the at least one acoustic sensor to determine a third maximum amplitude signal while the focused beam is rotated, the third maximum amplitude representing a third localization with respect to the acoustic axis.

22. The method of claim 21, further comprising locking the acoustic source at the position corresponding to the first maximum amplitude signal.

23. A guidewire comprising:
a core;
a coil coaxially disposed over the core;
a protective coating disposed on the coil;
at least one acoustic sensor disposed at a respective distance from a distal end of the shaft; and
at least one electrical conductor disposed in the protective coating, each electrical conductor electrically connecting a respective acoustic sensor to one or more electrical connection points in a housing attached to a proximal end of the guidewire.

24. The guidewire of claim 23, wherein each acoustic sensor comprises a piezoelectric polymer film disposed about at least a portion of a circumference of the core.

25. The guidewire of claim 23, wherein each acoustic sensor comprises a piezoelectric polymer film disposed about at least a portion of a circumference of the protective film.

26. The guidewire of claim 23, wherein each acoustic sensor comprises a piezoelectric polymer film disposed in the protective film.
